SEQUENCE LISTING (10 sequences)

SEQ ID NO: 1 - protein, 9 aa, Influenza A virus
GILGFVFTL

SEQ ID NO: 2 - protein, 9 aa, Artificial Sequence
  Optimised epitope sequence.
  MISC_FEATURE (1)..(9): D-amino acid isoform
LTFVFGLIG

SEQ ID NO: 3 - protein, 9 aa, Artificial Sequence
  Optimised epitope sequence.
  MISC_FEATURE (1)..(9): D-amino acid isoform
GPPQWNNPP

SEQ ID NO: 4 - protein, 9 aa, Artificial Sequence
  Optimised epitope sequence.
  MISC_FEATURE (1)..(9): D-amino acid isoform
RFPQWNNPP

SEQ ID NO: 5 - protein, 9 aa, Artificial Sequence
  Optimised epitope sequence.
  MISC_FEATURE (1)..(9): D-amino acid isoform
RFPQGNNPP

SEQ ID NO: 6 - protein, 9 aa, Artificial Sequence
  Optimised epitope sequence.
  MISC_FEATURE (1)..(9): D-amino acid isoform
RFPPWNNPP

SEQ ID NO: 7 - protein, 9 aa, Artificial Sequence
  Optimised epitope sequence.
  MISC_FEATURE (1)..(9): D-amino acid isoform
GPPPWNNPP

SEQ ID NO: 8 - protein, 9 aa, Artificial Sequence
  Optimised epitope sequence.
  MISC_FEATURE (1)..(9): D-amino acid isoform
GPPPGNNPP

SEQ ID NO: 9 - protein, 9 aa, Artificial Sequence
  Optimised epitope sequence.
  MISC_FEATURE (1)..(9): D-amino acid isoform
RFPPGNNPP

SEQ ID NO: 10 - protein, 9 aa, Artificial Sequence
  Optimised epitope sequence.
  MISC_FEATURE (1)..(9): D-amino acid
GPPQGNNPP

(19) United States
(12) Patent Application Publication
(10) Pub. No.: US 2020/0123198 A1
(43) Pub. Date: Apr. 23, 2020

(54) Peptide Libraries and Methods of Use

(71) Applicant: The Council of the Queensland Institute of Medical Research, Queensland (AU)

(72) Inventors: Andrew Sewell, South Glamorgan (GB); John Miles, Trinity Beach (AU)

(73) Assignee: The Council of the Queensland Institute of Medical Research (QIMR), Queensland (AU)

(21) Appl. No.: 16/339,189

(22) PCT Filed: Oct. 3, 2017
(86) PCT No.: PCT/AU2017/051077

§ 371 Date: Apr. 3, 2019

(30) Foreign Application Priority Data
Oct. 4, 2016 (AU) 2016904024

(51) Int. Cl. C07K 7/06; C07K 1/04; G01N 33/68; A61P 31/16; A61K 9/00

(52) U.S. Cl. CPC C07K 7/06; C07K 1/047; G01N 33/6863; A61P 31/16; A61K 38/00; G01N 2333/56; G01N 2333/55; G01N 2333/523; G01N 2333/70596; A61K 9/0053

(57) ABSTRACT

Disclosed are methods for identifying immunogenic peptides, and tools and/or reagents to be used in those methods. More specifically, the invention relates to combinatorial peptide library screening for synthetic antigenic peptides recognized by natural T-cell receptors.

This application is a national phase entry pursuant to 35 U.S.C. § 371 of International Application No. PCT/AU2017/051077, filed 3 Oct. 2017, which claims priority to Australian Provisional Application No. 2016904024 entitled “Peptide libraries and methods of use” filed 4 Oct. 2016, the contents of which are incorporated herein by reference in their entirety.

### FIELD OF THE INVENTION

The present application is filed with a Sequence Listing in electronic format. The Sequence Listing is provided as a file entitled “2019-10-24_01238-0001-00US_Seq_List.txt” created on Oct. 24, 2019, which is 4,096 bytes in size. The information in the electronic format of the sequence listing is incorporated herein by reference in its entirety.

This invention relates generally to methods for identifying immunogenic peptides, and tools and/or reagents to be used in those methods. More specifically, the invention relates to combinatorial peptide library screening for synthetic antigenic peptides recognized by natural T-cell receptors.

### BACKGROUND OF THE INVENTION

CD8⁺ T-cells recognize short peptide fragments presented by major histocompatibility complex class I (MHC-I) molecules on the surface of nucleated cells (see, Yewdell and Haeryfar, 2005; Yewdell, 2010; and Miles et al., 2015). These peptide-MHC-I (pMHC-I) molecular arrays are scanned by clonotypically distributed αβ T-cell receptors (TCRs) (see, Miles et al., 2011), which trigger T-cell activation beyond a preset monomeric TCR/pMHC-I affinity threshold (see, any of Bridgeman et al., 2012; Tan et al., 2015; Stepanek et al., 2014; van den Berg et al., 2013). This process enables the immune system to identify and eliminate infected and abnormal cells via targeted cytotoxicity, while remaining inert in the presence of healthy cells expressing a repertoire of unaltered self-derived peptides. Attenuated whole organisms, protein subunits and/or peptides are typically used in vaccine formulations to prime immune responses against various cancers and dangerous pathogens. In the setting of infectious disease alone, prophylactic vaccines are thought to prevent approximately 9 million deaths annually (see, UNICEF, 1996). However, effective prophylaxis is lacking for most human diseases and the global economic cost of current operational vaccines is high, costing around US$4 billion annually (see, Wolfson et al., 2006). In particular, the temperature-controlled supply chain for these sensitive biological compounds can account for up to 80% of the total deployment costs (see, Chen et al., 2011). Environmental stability is therefore a strategic priority for current vaccine research and development.

The vast majority of proteins in the natural world are constructed from L-amino acids, which are highly susceptible to degradation by endogenous and environmental proteases. In contrast, D-amino acids occur only rarely, for example in prokaryotic cell walls, bacterial antibiotics, certain animal proteins and venoms, as well as neuroregulators in the human brain (see, Pollegioni et al., 2007; Zhao and Lu, 2014; Martinez-Rodriguez et al., 2010; and Torres et al., 2005). Although D-amino acids are mirror image stereoisomers of L-amino acids with identical chemical and physical properties, the corresponding proteins are intrinsically resistant to protease-mediated hydrolysis (Zhao and Lu, 2014). Immunomodulating peptides designed from these building blocks may therefore allow the production of stable vaccines with enhanced bioavailability and in vivo efficacy. Additional benefits include potential for therapeutic activity via oral ingestion.

Large-scale T-cell scanning studies using combinatorial peptide libraries (CPLs) (Pinilla et al., 1999; Wooldridge, et al., 2012; and Ekeruche-Makinde et al., 2013) and yeast-displayed pMHC libraries (Birnbaum et al., 2014) have shown that cross-reactivity is an inherent property of TCRs (reviewed in Sewell, 2012). Accordingly, it may be feasible to generate non-natural D-amino acid agonists that mimic their native counterparts (Pentier et al., 2013; and Purcell et al., 2007). In work leading up to the present invention, a nonamer CPL was synthesized using only D-amino acid subunits to reverse engineer a fully synthetic agonist in the setting of a relevant human disease. The data validate a novel and systematic approach to the design of non-natural immunomodulatory compositions that offer substantial advantages over current vaccine formulations, as described hereafter.

### SUMMARY OF THE INVENTION

The present invention is predicated in part on the discovery that D-amino acid peptide “mimics” can effectively and specifically amplify human influenza-specific CD8⁺ T-cells in vitro, and initiate CD8⁺ T-cell responses in mice that are protective against lethal influenza challenge.

Furthermore, the present inventors have discovered that by interrogating whether a single defined D-amino acid at an index position in the context of a peptide of a defined length is recognized by a TCR, important information is obtained with respect to the degeneracy of each position of the peptide. Random amino acids at the other positions (i.e., other than the index position) result in the provision of peptides that have D-amino acids that bind to the MHC and that are also recognized by the cognate TCR. By systematically screening each D-amino acid at the index position for every position in the peptide, a list of the recognized D-amino acids at every position of the peptide can be generated, based on the individual D-amino acids that were found to bind the antigen-binding cleft of the MHC molecule and/or be recognized by the cognate TCR.

Even in the absence of actually characterizing any of the D-amino acids present in the random positions of any given peptide, the systematic approach allows for an assessment to be made on level of recognition by the TCR of the single fixed D-amino acid that is present at the index position being interrogated. Not all amino acids in a peptide will make connections with the MHC and/or TCR. Therefore, some positions may be variable with respect to the amino acid choice at a given position.

Surprisingly, the D-amino acid sequences of the synthetic agonists identified by the present inventors are significantly different from the native L-amino acid agonists and could not, therefore, have been predicted. Thus, the results presented herein reveal the power of CPLs in identifying agonistic D-amino acid peptide sequences in a systematic and unbiased fashion. The present invention has a clear and significant utility as a platform that is applicable to any target within the MHC (e.g., MHC class I) system, and can be used, for example, for rational design of effective human T-cell therapeutics that are potentially cheaper and more biologically stable than current formulations.

Accordingly, in one aspect the present invention provides a CPL for identifying synthetic T-cell receptor (TCR) agonists (i.e., D-amino acid peptides). These CPLs generally comprise a plurality of peptide sets, each set interrogating a different amino acid position (“index position”) of the peptide and comprising a plurality of separate peptide mixtures, wherein each mixture has a defined D-amino acid (i.e., a, c, d, e, i, f, g, h, k, l, m, n, p, q, r, s, t, v, w, y, or modified forms thereof) at the index position, and with every other position being a random D-amino acid, wherein the number of sets is equal to the number of positions present in the peptides. In specific embodiments, the peptide mixture comprises an approximately equivalent representation of each amino acid at each position other than the index position.

In some embodiments, the D-amino acids in positions other than the index position exclude cysteine. Embodiments of this type may be advantageous due to the propensity of cysteine to polymerize.

In some embodiments, each D-amino acid peptide in the peptide mixture is present at approximately equimolar concentrations. This has the beneficial effect of each individual peptide being present within a peptide mixture in small amounts (e.g., around 5.5×10⁻⁶ nM) that are generally insufficient to elicit a detectable immune. Therefore, any detectable recognition of a peptide mixture by a TCR during a screening process can be considered to result from the combination of all peptides within a mixture and thus characteristic of the fixed D-amino acid at the index position (as this is the only common feature between all of the peptides.

In some embodiments, the D-amino acid peptides are of a length that is suitable for presentation by MHC molecules. Moreover, in preferred embodiments all of the peptides included in the CPL are of an equal length to one another. Suitably, the MHC molecule to which the CPL peptides are expected to bind is a class I MHC molecule (“MHC-I”) and accordingly, such peptides are from about 8 D-amino acids to about 11 D-amino acids in length (i.e., 8, 9, 10, or 11 D-amino acids in length). In specific embodiments of the invention, the CPL comprises nonamers.

The CPL of the present invention can be synthesized using any means known in the art. For example, in some embodiments the peptides are synthesized by solid phase peptide synthesis (SPPS), microchip-based production, laser-based transfer of monomers in solid matrix, or any other suitable technique known in the art.

In some embodiments, each peptide mixture of the CPL is present in separate and distinct compartments of a container, or located to different containers. One advantage of each peptide mixture being present in a container, or compartment of a container, is the ease of facilitating high-throughput screening methods. By way of an illustrative example, each peptide mixture may be present in a separate well of a multi-well plate (e.g., a 96-well plate).

In some embodiments, the peptide mixtures are contacted with MHC molecules, such that each MHC molecule presents an individual peptide within the peptide mixture. As would generally be appreciated in the art, peptides are presented to effector cells (e.g., T-cells) by MHC molecules located on the surface of an antigen-presenting cell (APC). Thus, in some embodiments the peptides of the CPL are incubated with MHC molecules (e.g., MHC-I molecules) prior to exposure to the effector cell. In specific embodiments, the peptide mixtures of the CPL are contacted by the MHC-I at a higher concentration than the MHC-I molecules. The level of peptide loading into the antigen-binding cleft is significantly increased by providing a higher concentration of D-amino acid peptide as compared to the concentration of MHC-I molecules.

In another aspect, the present invention provides methods for identifying a T-cell peptide agonist capable of eliciting an immune response to a target antigen, the method comprising:
providing a combinatorial peptide library (CPL) to a MHC class I molecule known to interact with a T-cell receptor (TCR) of interest, the CPL comprising a plurality of peptide sets, each set interrogating a different amino acid position (“index position”) of the peptide and comprising a plurality of separate peptide mixtures, wherein each mixture has a defined D-amino acid (i.e., a, c, d, e, i, f, g, h, k, l, m, n, p, q, r, s, t, v, w, y, or modified forms thereof) at the index position, and with every other position being a random D-amino acid, wherein the number of sets is equal to the number of positions present in the peptides;
contacting each MHC and peptide mixture with a TCR known to bind the target antigen, to determine the D-amino acids recognized at each index position of the peptide;
generating an amino acid sequence by selecting recognized D-amino acids at each index position, to thereby identify TCR agonists that elicit or enhance an immune response to the target antigen.

An advantage provided by the present invention is that peptides identified by the screening methods described above and elsewhere herein have a higher stability, longer bioavailability, and/or slower elimination half-life than natural L-amino acid peptide agonists. Furthermore, D-amino acid peptides could not be predicted solely by analyzing the sequence of known L-amino acid peptide agonists, or by simply analyzing a structural model of relevant pMHC-TCR complexes.

In some embodiments, the screening method is a cell-based assay measuring an interaction between a target cell (e.g., APC) and an antigen-specific effector cell (e.g., antigen-specific T-cells). In specific embodiments of this type, the MHC molecule is present on the cell surface of an APC. In some of the same and other embodiments, the TCR is present on the surface of an effector cell. The effector cell can be any cell that is known to specifically recognize the target antigen in the context of an MHC. Non-limiting examples of suitable effector cells include antigen-specific cytotoxic T lymphocytes (CTL), and natural killer (NK) cells. Non-cell based assays are also contemplated (e.g., surface plasmon resonance (SPR) measurements of binding between pMHC and a TCR).

In some embodiments, the total concentration of the peptide mixture that is exposed to the TCR is between about 1 μM and about 500 μM. In some embodiments, the peptide mixture is contacted with the TCR at a concentration of about 100 μM.

In order to determine the level of recognition of each D-amino acid at an index position, an analysis of effector cell function is performed. For example, suitable analyses of effector cell function include cytotoxicity assays, and the measurement of cytokine and/or chemokine release from an effector cell (e.g., antigen-specific T-cell).

Several techniques to measure cytokine and/or chemokine release by cells are known in the art. However, such techniques include ELISA, ELISPOT, MHC-peptide tetramer staining, or flow-based analysis (e.g., flow cytometry). Any cytokine and/or chemokine that is known to be secreted from effector cells, particularly those closely associated with a Th1 immune response, is suitable for measuring the level of recognition of a candidate D-amino acid peptide. For example, suitable cytokines and/or chemokines may be selected from the group comprising MIP-1α, MIP-1β, IFN-γ, IL-1, IL-2, IL-8, IL-12, IL-18, TFNβ, CD107a, and RANTES.

A particularly suitable cytotoxicity assay for measuring effector cell function is a chromium release assay.

In yet some other embodiments of this type, the screening method further comprises the step of confirming that the identified peptides have the expected activity/immunogenicity. This confirmatory testing may be by way of any of the activity assays described above or elsewhere herein, or by any known method for assessing the presence of an immune response.

In still yet another aspect, the present invention provides immunogenic peptides that comprise, consist of, or consist essentially of a D-amino acid sequence represented by formula I:

X₁X₂pX₃X₄nnpp (I)

wherein:
X₁ is selected from g or r;
X₂ is selected from p or f;
X₃ is selected from p or q; and
X₄ is selected from w or g.

These D-amino acid peptides are of particular use in eliciting an immune response to the matrix M1 protein of influenza. Thus, in some embodiments these D-amino acid peptides can be used to treat an influenza virus infection. In specific embodiments, the D-amino acid sequence comprises the sequence set forth in any one of SEQ ID NO: 3-10. Preferably, the peptides of the present invention comprise the D-amino acid sequence set forth in SEQ ID NO: 3.

In a related aspect, the present invention provides an immunomodulating composition for eliciting or enhancing an immune response to the influenza matrix M1 protein. The compositions of this type comprise a D-amino acid peptide that comprises, consists of, or consists essentially of, an amino acid sequence represented by formula I:

X₁X₂pX₃X₄nnpp (I)

wherein:
X₁ is selected from g or r;
X₂ is selected from p or f;
X₃ is selected from p or q; and
X₄ is selected from w or g.

In specific embodiments of this type, the peptide comprises the D-amino acid sequence set forth in any one of SEQ ID NO: 3-10. Preferably, the D-amino acid sequence comprises the sequence set forth in SEQ ID NO: 3.

The compositions of the invention may be administered by any means known in the art. By way of an non-limiting example, the peptide may suitably be in particulate form.

In another related aspect, the present invention provides a pharmaceutical composition comprising a composition that comprises the D-amino acid sequence set forth in any one of SEQ ID NO: 3-10, together with a pharmaceutically acceptable carrier, diluent and/or excipient.

In some embodiments, the composition is formulated as a vaccine. One advantage of the present invention is that the present inventors have shown herein that peptides comprising D-amino acid sequences are more stable than L-amino acid-based peptides, and will therefore benefit from an increased shelf-life and more easily meet storage requirements.

In some embodiments, the composition is formulated for systemic administration. For example, the pharmaceutical composition may be formulated for oral administration. Surprisingly, it has been shown herein that D-amino acid peptides are much more stable than L-amino acid-based counterparts, and particularly in gastric-acid like conditions. Furthermore, it was also identified that D-amino acid peptides delivered orally retain the ability to prime effector T-cells within the gastrointestinal system without the need for an adjuvant. In specific embodiments, the D-amino acid peptides are formulated for oral or enteral administration. This presents an advantage over traditional L-amino acid peptide antigens, as it allows for direct contact of immunomodulating compositions with mucosal membranes such as the gastrointestinal tract. Furthermore, these administration routes reduce the risk of secondary infection caused from breaching the skin (and therefore particularly advantageous for livestock subjects).

In another aspect, the present invention provides methods for treating influenza in a subject, the method comprising administering a immunogenic D-amino acid peptide to a subject, wherein the peptide comprises, consists of, or consists essentially of an amino acid sequence represented by formula I:

X₁X₂pX₃X₄nnpp (I)

wherein:
X₁ is selected from g or r;
X₂ is selected from p or f;
X₃ is selected from p or q; and
X₄ is selected from w or g.

In specific embodiments, the D-amino acid sequence comprises, consists, or consists essentially the sequence set forth any one of SEQ ID NO: 3-10.

### BRIEF DESCRIPTION OF THE DRAWINGS

FIG. 1 is a schematic of a nine-residue long (9-mer) combinatorial peptide library. A nonamer (9-mer) library consists of 180 different peptide mixtures, each with one of the 20 D-amino acids in a defined position (index “O”) and all the amino acids except cysteine (c) in each of the remaining positions (“X”).

FIG. 2 shows a graphical representation and ELISPOT data of immunogenicity of the influenza retro-inverso and activity of the synthetic agonist via oral delivery. (A) Clonal ALF3 CD8⁺ T-cells were incubated with C1R-A2 target cells in the presence of GIL or ltf (10⁻⁵ M). MIP-1β release in the supernatants was quantified by ELISA. Error bars represent two replicates. (B) HLA-A2⁺ T2 cells were incubated with the indicated concentrations of GIL, ltf or EVDPIGHLY (an HLA-A1-restricted negative control) and stained with αHLA-A2-FITC. DMSO was included as an additional control. Mean fluorescence intensities are shown for each condition. (C) HHD mice were primed (day 0) and boosted (day 7 and day 14) via oral gavage with 100 μg of gpp or an equivalent volume of PBS (mock). Cells from mesenteric lymph nodes harvested on day 21 were stimulated for 10 days in vitro with GIL (10⁻⁶ M). GIL-reactive cells were then quantified in IFN-γ ELISPOT assays.

FIG. 3 shows that an archetypal human CD8⁺ T-cell clone has a wide recognition footprint across D-amino acid peptides. The ALF3 CD8⁺ T-cell clone cells was incubated with C1R-A2⁺ target cells coated with CPL arrays screens comprising of nonamer D-amino acids. Supernatant was harvested and MIP-1β release quantified by ELISA. The amino acid residue in each position corresponding to the index GIL peptide is depicted in red. Fixed amino acid positions (single letter code) along the peptide backbone are indicated. Error bars for individual peptide mixtures.

FIG. 4 provides a graphical representation illustrating that a fully synthetic T-cell agonist can be built using the CPL array that is highly resistant to human proteases and gastric acid. (A) The ALF3 CD8⁺ T-cell clone was incubated with C1R-A2⁺ target cells coated with eight D-amino acid candidate agonists, as suggested by quantitative signals in CPL peptide mixtures in logarithmically decreasing doses from 10⁻⁴ M to 10⁻⁷ M. Supernatant from the cultures were then harvested and MIP-1β release competitively quantified by MIP-1β ELISA. Error bars are shown. (B) The native L-peptide GIL and most optimal D-agonist gpp were added to human serum or MilliQ water with time course sampling from 0-60 min in triplicate. Ion peak signals that identified each agonist were then quantified using LCMS. The stability at each time point was calculated as the area of each serum treated ion peak as a percentage of the area of the same ion peak at 0 min (C) The native L-peptide GIL and most optimal D-agonist gpp were added to simulated gastric acid (NaCl, pepsin and HCL at pH 1.2) with time course sampling from 0-180 min in triplicate. Ion peak signals that identified each agonist were then quantified using LCMS. The stability at each time point was calculated as the area of each serum treated ion peak as a percentage of the area of the same ion peak at 0 mins. Error bars are shown.

FIG. 5 provides a graphical representation showing that a synthetic T-cell agonist activates influenza matrix epitope-specific CTLs in the context of HLA-A2 and is dependent on the CD8 co-receptor. (A) The CTL clone ALF3 was incubated with C1R-A2⁺ targets with GIL or gpp and MIP-1β release was quantified by ELISA after overnight incubation. Results show that gpp stimulates ALF3 CD8⁺ T-cell clone in a dose dependent fashion (B) The CTL clone ALF3 was incubated with GIL- or gpp-pulsed HLA-A2⁺ T2 targets and specific target cell killing was quantified by chromium release assay after a 4 hour incubation. Results show the ability of the CTL to lyse target cells in the coated in gpp in a dose dependent fashion (C) HLA-A2⁺ T2 cells were incubated with 10⁻⁴ M gpp, GIL, EVDPIGHLY (HLA-A1 binding negative control) or DMSO and subsequently stained with anti-HLA-A2-FITC antibody to quantify binding strength of peptide to surface HLA-A2 molecules. Shown are histograms for each peptide and control with the mean fluorescence intensity stated. (D) The CTL clone ALF3 was incubated overnight with 10⁻⁴ M gpp pulsed HLA-A2⁺C1R (A2+C1R), HLA-A2⁻ C1R (A2⁻ C1R), C1R-A2⁺ cells with enhanced CD8 binding (QE-C1R) and HLA-A2⁺ C1R cells that do not bind the CD8 co-receptor (227/8-C1R). MIP-1β in the supernatant was quantified by ELISA after overnight incubation. As shown, ALF3 cells were only activated in the context of HLA-A2⁺ and required CD8 co-receptor help.

FIG. 6 provides graphical representations showing that the D-amino acid peptide T-cell agonist is capable of stimulating cross-reactive T-cells with polyfunctional activity. The GIL-specific CTL clone GD (an analogous TRAV19⁺ clone to ALF3) was incubated with C1R-A2⁺ target cells pulsed with various concentrations of (A) GIL (B) gpp and (C) the irrelevant HLA-A2-restricted peptide, ELAGIGILTV (ELA), and assessed for five effector functions simultaneously (CD107a, IFN-γ, IL-2, MIP-1β and TNFα) using intracellular cytokine staining. Bars depict the percentage of CTL cells expressing each of the five functions measured when stimulated with 10⁻⁴ M (black bars) or 10⁻⁵ M (grey bars) of agonist (D) Pie charts depict the proportion of CTL cells performing the number of functions ranging from five functions (red), four functions (orange), three functions (yellow), two functions (green), one function (blue) and to no functions (grey) in response to agonist stimulation at 10⁻⁴ M or 10⁻⁵ M

FIG. 7 shows graphical representations that the synthetic T-cell agonist primes similar numbers of memory effectors with overlapping TCR repertoires. (A) Healthy adult HLA-A2⁺ PBMC were primed with either GIL or gpp and cultured in vitro for 10 days. Antigen specific cells were quantified with a HLA-A2-GIL tetramer using flow cytometry. Percentages of total CD8⁺ cells are shown. The Fluorescence Minus One (FMO) control is shown. (B) GIL- or gpp-primed T-cell effectors were combined with ⁵¹Cr-labeled C1R-HLA-A2⁺ targets cells expressing the influenza M1 protein at a descending target-to-effector ratios. Target lysis was quantified using supernatant in a liquid scintillator (C) GIL- or gpp-primed T-cell effectors were combined with CFSE-labeled CIR-A2⁺ targets pulsed with 10⁻⁶ M GIL peptide. Target lysis was quantified using flow cytometry. HLA-A2-GIL tetramer positive cells from GIL- or gpp-primed T-cell cultures were sorted to high purity (>98%) and clonotyped using quantitative template-switch anchored TCR RT-PCR and Sanger sequencing. (D) The number of unique clonotypes were calculated for each experimental condition across each donor. Total TRBV usage (E) and TRBJ usage (F) for GIL- and gpp-primed T-cell across three healthy, genetically unrelated donors are shown. Total numbers of in frame clonotypes for each donor are shown. Random sequence sampling was performed to produce an equal numbers of clonotypes for each experimental condition across each donor. ns: not significant.

FIG. 8 provides a graphical model that demonstrates that the native and synthetic T-cell peptide agonists can form similar overall structural conformations. (A) Side view of the GIL peptide (orange sticks) within the HLA-A2 binding cleft (grey cartoon). Peptide residues highlighting the central bulge between positions 4 and 7 are shown. (B) The structure of the D-amino acid agonist gpp (blue sticks) within the HLA-A2 binding cleft (grey cartoon) was modelled using the JM22-HLA-A2-GIL complex (PDB: IOGA) using Wincoot. (C) Superposition of GIL peptide (orange sticks) and gpp agonist (blue sticks) presented by the HLA-A2 molecule (grey cartoon). Arrows demonstrate the main TCR contact points based on the JM22-HLA-A2-GIL complex at residues 4, 5, 6 and 8.

FIG. 9 provides graphical representation of the vaccination of humanized mice with the synthetic T-cell agonist protection against lethal influenza virus infection. (A) HHD mice were primed (day 0) and boosted (day 14) through subcutaneous injection with either 100 μg of GILGFVFTL (GIL, n=4), gppqwnnpp (gpp, n=4) or with DMSO (mock, n=2) and 100 μL Incomplete Freunds Adjuvant. Single cell suspensions were generated from spleens and peripheral lymph nodes (LN) extracted on day 21. GIL-specific cells were detected by ELISPOT, where cells were incubated overnight with 10⁻⁵ M GIL and the numbers of cells producing IFN-γ were numerated. The number of spot-forming units (SFU) was normalized through deducting background obtained from no peptide stimulation controls. Representative ELISPOT wells are shown under each bar. Results shown are of a single experiment which was repeated with similar results. (B) HHD mice were vaccinated subcutaneously with 100 μg of GIL (n=17), gpp (n=20) or an irrelevant L-amino acid peptide (ELAGIGILTV, ELA, n=17) and 100 μL Incomplete Freunds Adjuvant, and boosted with the same formula on day 14. A separate group of mice were not given any injections (not vac, n=7). The mice were infected with influenza strain H1N1 A/PR/8/34 (PR8) on day 21 at 50 PFU for female mice and 100 PFU for male mice, and their weights monitored for the next 8 days. Mice that lost more than 20% of their initial body weight were deemed not to have survived the challenge and were euthanized. NS, not significantly different by Student's t test (p=0.4); * represents p=0.03; ** represents p=0.002. TABLE A

### BRIEF DESCRIPTION OF THE SEQUENCES

| SEQUENCE ID | | |
|---|---|---|
| NUMBER | SEQUENCE | LENGTH |
| SEQ ID NO: 1 | GILGFVFTL | 9 aa |
| SEQ ID NO: 2 | ltfvfglig | 9 aa |
| SEQ ID NO: 3 | gppqwnnpp | 9 aa |
| SEQ ID NO: 4 | rfpqwnnpp | 9 aa |
| SEQ ID NO: 5 | rfpqgnnpp | 9 aa |
| SEQ ID NO: 6 | rfppwnnpp | 9 aa |
| SEQ ID NO: 7 | gpppwnnpp | 9 aa |
| SEQ ID NO: 8 | gpppgnnpp | 9 aa |
| SEQ ID NO: 9 | rfppgnnpp | 9 aa |
| SEQ ID NO: 10 | gppqgnnpp | 9 aa |

### DETAILED DESCRIPTION OF THE INVENTION

### 1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

By “about” or “approximate” and their grammatically equivalent expressions is meant a quantity, level, value, number, frequency, percentage, dimension, size, amount, abundance, concentration, weight or length that varies by as much 15, 14, 13, 12, 11, 10, 9, 8, 7, 6, 5, 4, 3, 2 or 1% to a reference quantity, level, value, number, frequency, percentage, dimension, size, amount, abundance, concentration, weight or length.

The term “TCR agonist” as used herein refers to any agent which directly or indirectly agonizes or antagonizes a component so as to agonize or otherwise activate or increase the function of a TCR. In some embodiments, the TCR agonist directly agonizes TCR function. Suitably, a D-amino acid peptide antigen may directly agonize TCR function by binding with the TCR and activating the receptor

By “antigen” is meant all, or part of, a molecule (e.g., a protein, peptide, or other molecule or macromolecule) capable of being bound by an antibody or a T-cell receptor (TCR) if presented by MHC molecules An antigen may be additionally capable of being recognized by the immune system and/or being capable of stimulating or inducing a humoral immune response and/or cellular immune response leading to the activation of B- and/or T-lymphocytes. An antigen may have one or more epitopes (B- and T-epitopes). Antigens as used herein may also be mixtures of several individual antigens.

As used herein, the term “bioavailability” refers to the systemic availability of a given amount of a particular peptide administered to a subject. Bioavailability is an absolute term that indicates measurement of both the time (rate) and total amount (extent) of peptide that reaches the general circulation from an administered dosage form. Bioavailability may be assessed by measuring the area under the curve (AUC) or the maximum serum or plasma concentration (Cmax) of the unchanged form of a peptide following administration of the compound to a mammal. AUC is a determination of the area under the curve plotting the serum or plasma concentration of a compound along the ordinate (Y-axis) against time along the abscissa (X-axis). Generally, the AUC for a particular compound may be calculated using methods known to those of ordinary skill in the art and as described in G. S. Banker, Modern Pharmaceutics, Drugs and the Pharmaceutical Sciences, v. 72, Marcel Dekker, New York, Inc., 1996, the contents of which are herein incorporated by reference in their entirety.

Throughout this specification, unless the context requires otherwise, the words “comprise”, “comprises” and “comprising” will be understood to imply the inclusion of a stated step or element or group of steps or elements but not the exclusion of any other step or element or group of steps or elements. Thus, use of the term “comprising” and the like indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

By “effective amount”, in the context of modulating an immune response or treating or preventing a disease or condition, is meant the administration of that amount of composition to an individual in need thereof, either in a single dose or as part of a series, that is effective for that modulation, treatment or prevention. The effective amount will vary depending upon the health and physical condition of the individual to be treated, the taxonomic group of individual to be treated, the formulation of the composition, the assessment of the medical situation, and other relevant factors. It is expected that the amount will fall in a relatively broad range that can be determined through routine trials.

As used herein, the term “effector cell” means an immune cell which is involved in the effector phase of an immune response, as opposed to the cognitive and activation phases of an immune response. Exemplary immune cells include a cell of a myeloid or lymphoid origin, e.g., lymphocytes (e.g., T-cells including cytolytic T cells (CTLs)), killer cells, natural killer cells, monocytes, eosinophils, neutrophils, polymorphonuclear cells, granulocytes, mast cells, and basophils. Effector cells express specific Fc receptors and carry out specific immune functions. An effector cell can induce antibody-dependent cell-mediated cytotoxicity (ADCC), e.g., a neutrophil capable of inducing ADCC. For example, monocytes, macrophages, neutrophils, eosinophils, and lymphocytes which express FcαR are involved in specific killing of target cells and presenting antigens to other components of the immune system, or binding to cells that present antigens. An effector cell can also phagocytose a target antigen, target cell, metastatic cancer cell, or microorganism.

The term “elimination half-life” as used herein refers to the terminal log-linear rate of elimination of a peptide from the plasma of a subject. Those of skill in the art will appreciate that half-life is a derived parameter that changes as a function of both clearance and volume of distribution. The terms “extended”, “longer”, or “increased” used in the context of elimination half-life are used interchangeably herein and are intended to mean that there is a statistically significant increase in the half-life of a peptide (e.g., a candidate D-amino acid peptide) relative to that of the reference molecule (e.g., an L-amino acid peptide) as determined under comparable conditions.

As used herein, the term “immunomodulatory composition” refers to a composition or formulation which contains the composition of the present invention and which is in a form that is capable of being administered to any vertebrate, preferably an animal, preferably a mammal, and more preferably a human. Typically, the immunomodulatory composition includes or is prepared from dry powder in a conventional saline or buffered aqueous solution medium in which the composition of the present invention is suspended or dissolved. In this form, the composition of the present invention can be used conveniently to prevent, ameliorate, manage or otherwise treat a condition. Upon introduction into a host, the immunomodulatory composition is able to elicit an immune response, preferably a detectable immune response, including, but not limited to, the production of antibodies, cytokines and/or the activation of cytotoxic T-cells, antigen presenting cells, helper T-cells, dendritic cells and/or other cellular responses. The immunomodulatory composition of the present invention includes influenza peptide sequences, preferably conserved influenza peptide sequences, and more preferably repeated conserved influenza peptide sequences. Immunomodulating compositions of the present invention may include or be administered in or with an adjuvant.

In the context of the invention, the term “loading” means the assembly of a peptide in an antigen-binding cleft of an MHC molecule.

By “obtained from” is meant that a sample such as, for example, a polynucleotide extract or polypeptide extract is isolated from, or derived from, a particular source.

The terms “patient”, “subject”, “host” or “individual” used interchangeably herein, refer to any subject, particularly a vertebrate subject, and even more particularly a mammalian subject, for whom therapy or prophylaxis is desired. Suitable vertebrate animals that fall within the scope of the invention include, but are not restricted to, any member of the subphylum Chordata including primates (e.g., humans, monkeys and apes, and includes species of monkeys such from the genus Macaca (e.g., cynomologus monkeys such as Macaca fascicularis, and/or rhesus monkeys (Macaca mulatta)) and baboon (Papio ursinus), as well as marmosets (species from the genus Callithrix), squirrel monkeys (species from the genus Saimiri) and tamarins (species from the genus Saguinus), as well as species of apes such as chimpanzees (Pan troglodytes)), rodents (e.g., mice rats, guinea pigs), lagomorphs (e.g., rabbits, hares), bovines (e.g., cattle), ovines (e.g., sheep), caprines (e.g., goats), porcines (e.g., pigs), equines (e.g., horses), canines (e.g., dogs), felines (e.g., cats), avians (e.g., chickens, turkeys, ducks, geese, companion birds such as canaries, budgerigars etc.), marine mammals (e.g., dolphins, whales), reptiles (snakes, frogs, lizards etc.), and fish. A preferred subject is a human in need of stimulating or inducing an antigen-specific Th2 response, suppressing the development of an antigen-specific Th1 response, stimulating the development in antigen-presenting cells of an alternatively activated phenotype, preventing or inhibiting the activation of antigen-presenting cells by an inflammatory stimulus, binding to lipopolysaccharide, preventing or inhibiting binding of lipopolysaccharide to lipopolysaccharide-binding protein, preventing or inhibiting binding of toll-like receptor (TLR) ligands (e.g., lipopolysaccharide) to antigen-presenting cells, interacting with the plasma membrane of antigen-presenting cells, and down-regulating or impairing lysosome function in antigen-presenting cells, or in need of treatment or prophylaxis of an undesirable or deleterious immune response, including autoimmune diseases, allergies and transplantation associated diseases, which are often associated with the presence or aberrant expression of an antigen of interest. However, it will be understood that the aforementioned terms do not imply that symptoms are present.

The term “peptide mixture” as used herein refers to an admixture of different peptides. Typically, the peptides within the admixture have at least one feature in common, for example, all having a fixed D-amino acid at a particular position of the peptide. The peptide mixture typically comprises a sufficient number of peptides that spans the variation of amino acid at each position of the peptide other then the fixed position. The identity of individual peptides within the peptide mixture does not need to be known or fully characterized, especially when using the peptide mixture to interrogate a feature that is uniform between all peptides included in the peptide mixture.

The terms “peptide set”, “set” or the like, as used herein, refer to a plurality of separate peptide compositions which are grouped together for the purposes of performing a specific screening method. For example, a peptide set used to interrogate the recognition of different amino acids at a first position of an amino acid sequence, would typically comprise as many separate peptide compositions as amino acids being interrogated, and individual peptide compositions within the set would have a different amino acid at the first position of that sequence.

By “pharmaceutically acceptable carrier” is meant a solid or liquid filler, diluent or encapsulating substance that can be safely used in topical or systemic administration to an animal, preferably a mammal, including humans.

“Polypeptide”, “peptide”, “protein” and “proteinaceous molecule” are used interchangeably herein to refer to molecules comprising or consisting of a polymer of amino acid residues and to variants and synthetic analogues of the same. Thus, these terms apply to amino acid polymers in which one or more amino acid residues are synthetic non-naturally occurring amino acids, such as a chemical analogue of a corresponding naturally occurring amino acid, as well as to naturally-occurring amino acid polymers.

The term “target cell” as used herein, refers to any cell that binds a target antigen or a D-amino acid peptide T cell agonist in the antigen-binding grove of an MHC molecule (e.g., an APC, including macrophages, B-cells and dendritic cells).

The term “Th1” refers to a subclass of T helper cells that produce inter alia IL-1, IL-2, IL-8, IL-12, IL-18, interferon-gamma (IFN-γ), tumor necrosis factor-alpha (TNF-α), and which elicit inflammatory reactions associated with a cellular, i.e., non-immunoglobulin, response to a challenge. Thus, a Th1 cytokine response or T1 cytokine response encompasses an immune response whose most prominent feature comprises abundant CD4⁺ helper T-cell activation that is associated with increased levels of T1 cytokines (e.g., IL-1, IL-2, IL-8, IL-12, IL-18, IFN-γ, TNF-α, etc.) relative to these cytokine amounts in the absence of activation. A T1 cytokine response can also refer to the production of T1 cytokines from other white blood cells and nonwhite blood cells. A Th1 cytokine response can include abundant CD8 cytotoxic T lymphocyte activity including T1 cytokine production, referred to as Tc1. A Th1 response is typically promoted by CD4 “Th1” T-helper cells however a Th1 response can include CD8 Tc1 T cytotoxic cells.

As used herein, the terms “treatment”, “treating”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment”, as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

### 2. Abbreviations

The following abbreviations are used throughout the application:
nt=nucleotide
nts=nucleotides
aa=amino acid(s)
kb=kilobase(s) or kilobase pair(s)
kDa=kilodalton(s)
d=day
h=hour
s=seconds
APC=antigen-presenting cell
CPL=combinatorial peptide library
MHC=major histocompatibility complex
TCR=T-cell receptor

### 3. Combinatorial Peptide Libraries (CPL)

The present invention is based in part on the determination that novel D-amino acid peptides are as effective as a native L-amino acid peptide in generating functional T-cell responses, yet exhibit little (if any) sequence similarity. The present inventors realized that without adopting a rational screening method, D-amino acid peptides could not simply be identified by prediction. The present inventions thus set out to generate a D-amino acid peptide library that could be use to identify novel peptides that can generate a functional and antigen-specific T-cell response. It is considered that newly identified D-amino acid peptide T-cell agonists would be useful to treat any number of diseases wherein an enhanced immune response would be beneficial.

Accordingly, the present invention provides combinatorial peptide libraries (CPL) that are useful for screening for novel D-amino acid peptides for recognition by T-cell TCRs. The level of recognition can be characterized by any means described above, or in further detail below, including cellular immunogenicity, or the ability to lyse APC which present the T-cell-specific peptide in the context of an MHC molecule. By creating a complete CPL, a generally more robust approach as compared to a single amino acid substitution is achieved. The CPL of the present invention makes use of combinatorial chemistry to produce simultaneous large arrays of multiply-substituted peptides. The information obtained from CPL approaches for studying T-cell agonists as described above and elsewhere herein, can be combined with information gained from other assessments of peptide immunogenicity in order to identify D-amino acid peptides that are T-cell agonists.

The CPLs of the invention conveniently facilitate positional scanning-type CPL experiments. These methods provide a convenient and rational platform to screen a large number of D-amino acid peptides, and thus obtain information regarding the TCR recognition at each position of a D-amino acid peptide. Importantly, screening by positional scanning allows for a clear correlation to be made between changes in peptide sequence at specific positions of the peptide and associated change in immunogenicity.

An important attribute of CPL screening is the ability to simultaneously scan all possible D-amino acid combinations at all positions of a peptide sequence. This process may identify a number of more appropriate (better recognized) D-amino acids than a directed amino acid substitution specifically made at a single position. Recent studies utilizing the positional scanning format have identified high potency L-amino acid peptides which even exceed the binding affinity of the natural peptide. The focus of these previous reports has been either to discover new T-cell agonists, or enhance existing epitope immunogenicity specific to individual inbred mouse strains, or human cell clones (as described by Gundlach et al., (1996) J. Immunol. 156: 3645-3652; Hemmer et al., (1998) J. Immunol. 156: 3631-3636; Wilson et al., (1999) J. Immunol. 163: 6424-6434).

Preferably, the CPLs are composed of peptides of uniform length and having specific positions (“index positions”) systematically defined. A typical CPL comprises a peptide set for each position along the length of the peptide.

For example, an octamer (8-mer) CPL comprises eight independent peptide sets, and is represented as O₁XXXXXXX, XO₂XXXXXX, XXO₃XXXXX, XXXO₄XXXXX, XXXXO₅XXX, XXXXXO₆XX, XXXXXO₇X, XXXXXXXO₈, in which O₁-O₈ each represent a position occupied by a defined D-amino acid, and X represents positions occupied by a random D-amino acid.

In another example, a nonamer (9-mer) CPL comprises nine independent peptide sets, and is represented as O₁XXXXXXXX, XO₂XXXXXXX, XXO₃XXXXXX, XXXO₄XXXXX, XXXXO₅XXXX, XXXXXO₆XXX, XXXXXXO₇XX, XXXXXXXO₈X, XXXXXXXXO₉; in which O₁-O₉ each represent a position occupied by a defined D-amino acid, and X represents positions occupied by a random D-amino acid.

In yet another example, a decamer (10-mer) CPL comprises ten independent peptide set, and is represented as O₁XXXXXXXXX, XO₂XXXXXXXX, XXO₃XXXXXXX, XXXO₄XXXXXX, XXXXO₅XXXXX, XXXXXO₆XXXX, XXXXXXO₇XXX, XXXXXXXO₈XX, XXXXXXXXO₉X, XXXXXXXXXO₁₀ in which O₁-O₁₀ each represent a position occupied by a defined D-amino acid, and X represents positions occupied by a random D-amino acid.

Each peptide set in the CPL comprises a plurality of peptide mixtures, with each peptide mixture having a different D-amino acid occupying the “O” position. The screening of a CPL can provide information about the recognition of each different D-amino acid at each position of the candidate peptide by a T-cell clone.

3.1 Producing the CPL

Defining the length of the D-amino acid peptide that is recognized by a T-cell clone is a typical starting point in designing and/or selecting an appropriate CPL. For example, if a CPL was being constructed to identify a D-amino acid peptide to be recognized by the T-cell clone ALF3, then one would consider the length of the native epitope (i.e., GILGFVFTL₅₈₋₆₆ peptide from the Matrix M1 protein, SEQ ID NO: 1), which is a nonamer. Accordingly, in this illustrative example, a nonamer peptide CPL would be selected. A measureable cytotoxic activity from a peptide mixture indicates that the single defined fixed D-amino acid is recognized by the target antigen-specific TCR, and thereby triggers the desired T-cell response.

Once the peptide length of the CPL is determined, the library can be synthesized using any means known in the art. For example, in some embodiments the peptides are synthesized by a method selected from: solid phase peptide synthesis (exemplary methods of this type are described in Pedersen et al., (2012) Microwave heating in solid-phase peptide synthesis. Chem Soc Rev 41: 1826-1844), microchip-based production (using techniques similar to that described by Schirwitz et al., 2009), or laser-based transfer of monomers in solid matrix (such as that described by Loeffler et al., 2015). In light of recent developments of cell-free systems for producing combinatorial peptides, making use of expanded genetic codes bay be suitable for using with the present invention. Such methods are described, for example, by Taira et al, J Biosci Bioeng., 2005, 99 (5): 473-6. Therefore, in some embodiments, the CPL of the present invention may be prepared using ribozyme-based production methods (as generally described by Cui et al., 2004).

As T-cells recognize peptide antigens in the context of an MHC molecule, prior to performing any screening experiments with the CPL, the antigen-binding cleft of an appropriate MHC molecule may be loaded with a peptide that is present in the peptide mixtures of the CPL. Methods of loading peptides into an MHC antigen-binding cleft are well known in the art. The MHC molecule selected will be chosen based on the screening experiment to be performed. That is, the MHC molecule must be recognized by the cognate antigen-specific TCR for which D-amino acid peptide recognition is being assessed. In specific embodiments, the MHC molecule is selected from the group consisting of MHC class I (MHC-I), MHC class II (MHC-II), non-classical MHC, homo-oligomers thereof, hetero oligomers thereof, and mixtures of the same. The MHC molecules may be from any vertebrate species, e.g., primate species, particularly humans; rodents, including mice, rats, hamsters, and rabbits; equines, bovines, canines, felines; etc. Of particular interest are the human HLA molecules, and the murine H-2 molecules. Included in the HLA molecules are the MHC-I molecules HLA-A, HLA-B, HLA-C, and β2-microglobulin; and the MHC-II molecules include subunits HLA-DPα, HLA-DPβ, HLA-DQα, HLA-DQβ, HLA-DRα, and HLA-DRβ. Additionally, the murine H-2 molecules include the MHC-I H-2K, H-2D, H-2L, and the MHC-II I-Aα, I-Aβ, I-Eα, and I-Eβ, and β2-microglobulin. Amino acid sequences of some representative MHC molecules are referenced in European Patent No. EP812 331. Also included in the scope of this invention are non-classical molecules such as HLA-E, HLA-F, HLA-G, and Qa1.

In an exemplary method, an MHC molecule may be incubated with the peptide to be presented for about 4 hours to about 6 hours, or for about 2 hours to about 3 hours. For such an incubation, a total peptide mixture concentration of about 100 μM is suitable, however, this may vary. Generally, about 1 μM to about 500 μM can typically be used. Ideally, the concentration of the D-amino acid peptide is sufficient to saturate the MHC molecule, and thus adequately bind the peptide into the antigen-binding groove of the MHC molecule. However, many methods of loading peptides into the antigen-binding cleft of an MHC molecule are known in the art, any of which can be used with the present invention.

### 4. Methods of Identifying D-Amino Acid T-Cell Peptide Agonists

4.1 Incorporating the CPL into Screening Methods

In order to identify D-amino acid peptides that exhibit T-cell agonistic activity, the CPL can be incorporated into a method of screening. Accordingly, in specific embodiments, the present invention includes methods for identifying a T-cell peptide agonist capable of eliciting an immune response to a target antigen, the method comprising:
providing a CPL as described above and elsewhere herein to a MHC class I molecule known to interact with a T-cell receptor (TCR) of interest, the CPL comprising a plurality of peptide sets, each set interrogating a different amino acid position (“index position”) of the peptide and comprising a plurality of separate peptide mixtures, wherein each mixture has a single defined D-amino acid (i.e., a, c, d, e, i, f, g, h, k, l, m, n, p, q, r, s, t, v, w, y, or modified forms thereof) at the index position, and with every other position being a random D-amino acid, wherein the number of sets is equal to the number of positions present in the peptides;
contacting each MHC and peptide mixture with a TCR known to bind the target antigen, to determine the D-amino acids recognized at each index position of the peptide;
generating an amino acid sequence by selecting recognized D-amino acids at each index position, to thereby identify TCR agonists that elicit or enhance an immune response to the target antigen.

In some embodiments, the methods of the invention may be performed as cell-free assays. In these cell-free assay embodiments, the MHC molecule generally corresponds to the soluble form of the normally membrane-bound protein. For MHC-I subunits, the soluble form is derived from the native form by deletion of the transmembrane and cytoplasmic domains. In these embodiments the MHC will be typically produced recombinantly, before being refolded and purified in the presence of the D-amino acid peptide of interest (or peptide mixture). Standard production protocols for MHC-peptide complexes can be applied. These methods generally involve refolding insoluble MHC together with β₂-microglobulin (β₂m) and the peptide (or peptide mixture) in a suitable refolding buffer. The refolding buffer and protein mixture is then suitably concentrated to a more convenient volume for purification (for example, about 50 mL, about 30 mL, about 20 mL, about 10 mL, about 7 mL, about 5 mL or about 2 mL). The concentrated protein mixture can then be purified by size exclusion chromatography, and ion exclusion chromatography, as required. Other methods of loading peptide antigens into the antigen-binding cleft of MHC molecules are well known in the art, and any suitable method can be used. Notably, a detailed description of methods suitable for purifying MHC-I molecules is provided in International PCT Publication No. WO2006/103406, the disclosures of which are incorporated herein by reference.

In some embodiments, the MHC molecule is located on the cell surface on a target cell (e.g., an APC). By way of an illustrative example, T2 cells can be used as a suitable APC that expresses the HLA A2 MHC-I molecule. T2 cells lack the transporter associated with antigen processing (TAP) and therefore the addition of exogenous peptide antigen is required for stable cell surface expression of the MHC-I. For other cell lines, however, the APC may present an endogenous peptide in the MHC, which will require to be “stripped” our of the antigen-binding clef of the molecule. This can be performed by incubating the cells at a low pH (e.g., pH 2-3) for a short period, before reloading with a specific peptide or a peptide present in the peptide mixture.

4.1.1 Sources of APC and their Precursors

APC or their precursors can be isolated by methods known to those of skill in the art. The source of such cells will differ depending upon the APC required which is dependent on the nature of the function the D-amino acid peptide is expected to have. For example, the expected function of an identified peptide could be to elicit an immune response to the Matrix I protein from influenza. In this context, the APC can be selected from dendritic cells, macrophages, monocytes and other cells of myeloid lineage.

Typically, precursors of APC can be isolated from any tissue, but are most easily isolated from blood, cord blood or bone marrow (Sorg et al., 2001, Exp Hematol. 29, 1289-1294; Zheng et al., 2000, J Hematother Stem Cell Res. 9, 453-464). It is also possible to obtain suitable precursors from diseased tissues such as rheumatoid synovial tissue or fluid following biopsy or joint tap (Thomas et al., 1994a, J Immunol. 153, 4016-4028; Thomas et al., 1994b, Arthritis Rheum. 37(4)). Other examples include, but are not limited to liver, spleen, heart, kidney, gut and tonsil (Lu et al., 1994, J Exp Med. 179, 1823-1834; McIlroy et al., 2001, Blood 97, 3470-3477; Vremec et al., 2000, J Immunol. 159, 565-573; Hart and Fabre, 1981, J Exp Med. 154(2), 347-361; Hart and McKenzie, 1988, J Exp Med. 168(1), 157-170; Pavli et al., 1990, Immunology 70(1), 40-47).

Leukocytes isolated directly from tissue provide a major source of APC precursors. Typically, these precursors can only differentiate into APCs by culturing in the presence or absence of various growth factors. According to the practice of the present invention, the APCs may be so differentiated from crude mixtures or from partially or substantially purified preparations of precursors. Leukocytes can be conveniently purified from blood or bone marrow by density gradient centrifugation using, for example, Ficoll Hypaque which eliminates neutrophils and red cells (peripheral blood mononuclear cells or PBMCs), or by ammonium chloride lysis of red cells (leukocytes or white blood cells). Many precursors of APCs are present in peripheral blood as non-proliferating monocytes, which can be differentiated into specific APCs, including macrophages and dendritic cells, by culturing in the presence of specific cytokines.

Tissue-derived precursors such as precursors of tissue dendritic cells or of Langerhans cells are typically obtained by mincing tissue (e.g., basal layer of epidermis) and digesting it with collagenase or dispase followed by density gradient separation, or selection of precursors based on their expression of cell surface markers. For example, Langerhans cell precursors express CD1 molecules as well as HLA-DR and can be purified on this basis.

In some embodiments, the APC precursor is a precursor of macrophages. Generally these precursors can be obtained from monocytes of any source and can be differentiated into macrophages by prolonged incubation in the presence of medium and macrophage colony stimulating factor (M-CSF) (Erickson-Miller et al., 1990, Int J Cell Cloning 8, 346-356; Metcalf and Burgess, 1982, J Cell Physiol. 111, 275-283).

In other embodiments, the antigen presenting cell precursor is a precursor of Langerhans cells. Usually, Langerhans cells can be generated from human monocytes or CD34⁺ bone marrow precursors in the presence of granulocyte/macrophage colony-stimulating factor (GM-CSF), IL-4/TNF_{α} and TGF_{β} (Geissmann et al., 1998, J Exp Med. 187, 961-966; Strobl et al., 1997a, Blood 90, 1425-1434; Strobl et al., 1997b, dv Exp Med Biol. 417, 161-165; Strobl et al., 1996, J Immunol. 157, 1499-1507).

In still other embodiments, the APC precursor is a precursor of dendritic cells. Several potential dendritic cell precursors can be obtained from peripheral blood, cord blood or bone marrow. These include monocytes, CD34⁺ stem cells, granulocytes, CD33⁺CD11c⁺ dendritic cell precursors, and committed myeloid progenitors—described below.

Monocytes:

Monocytes can be purified by adherence to plastic for 1-2 hours in the presence of tissue culture medium (e.g., RPMI) and serum (e.g., human or foetal calf serum), or in serum-free medium (Anton et al., 1998, Scand J Immunol. 47, 116-121; Araki et al., 2001, Br J Haematol. 114, 681-689; Mackensen et al., 2000, Int J Cancer 86, 385-392; Nestle et al., 1998, Nat Med. 4, 328-332; Romani et al., 1996, J Immunol Meth. 196, 137-151; Thurner et al., 1999, J Immunol Methods 223, 1-15). Monocytes can also be elutriated from peripheral blood (Garderet et al., 2001, J Hematother Stem Cell Res. 10, 553-567). Monocytes can also be purified by immunoaffinity techniques, including immunomagnetic selection, flow cytometric sorting or panning (Araki et al., 2001, supra; Battye and Shortman, 1991, Curr. Opin. Immunol. 3, 238-241), with anti-CD14 antibodies to obtain CD14hi cells. The numbers (and therefore yield) of circulating monocytes can be enhanced by the in vivo use of various cytokines including GM-CSF (Groopman et al., 1987, N Engl J Med. 317, 593-598; Hill et al., 1995, J Leukoc Biol. 58, 634-642). Monocytes can be differentiated into dendritic cells by prolonged incubation in the presence of GM-CSF and IL-4 (Romani et al., 1994, J Exp Med. 180, 83-93; Romani et al., 1996, supra). A combination of GM-CSF and IL-4 at a concentration of each at between about 200 to about 2000 U/mL, more preferably between about 500 to about 1000 U/mL and even more preferably between about 800 U/mL (GM-CSF) and 1000 U/mL (IL-4) produces significant quantities of immature dendritic cells, i.e., antigen-capturing phagocytic dendritic cells. Other cytokines which promote differentiation of monocytes into antigen-capturing phagocytic dendritic cells include, for example, IL-13.

CD34⁺ Stem Cells:

Dendritic cells can also be generated from CD34⁺ bone marrow derived precursors in the presence of GM-CSF, TNFα±stem cell factor (SCF, c-kitL), or GM-CSF, IL-4±flt3L (Bai et al., 2002, Int J Oncol. 20, 247-53; Chen et al., 2001, Clin Immunol. 98, 280-292; Loudovaris et al., 2001, J Hematother Stem Cell Res. 10, 569-578). CD34+ cells can be derived from a bone marrow aspirate or from blood and can be enriched as for monocytes using, for example, immunomagnetic selection or immunocolumns (Davis et al., 1994, J Immunol Meth. 175, 247-257). The proportion of CD34⁺ cells in blood can be enhanced by the in vivo use of various cytokines including (most commonly) G-CSF, but also flt3L and progenipoietin (Fleming et al., 2001, Exp Hematol. 29, 943-951; Pulendran et al., 2000, J Immunol. 165, 566-572; Robinson et al., 2000, J Hematother Stem Cell Res. 9, 711-720).

Other Myeloid Progenitors:

Dendritic cells can be generated from committed early myeloid progenitors in a similar fashion to CD34⁺ stem cells, in the presence of GM-CSF and IL-4/TNF. Such myeloid precursors infiltrate many tissues in inflammation, including rheumatoid arthritis synovial fluid (Santiago-Schwarz et al., 2001, J Immunol. 167, 1758-1768). Expansion of total body myeloid cells including circulating dendritic cell precursors and monocytes, can be achieved with certain cytokines, including flt-3 ligand, granulocyte colony-stimulating factor (G-CSF) or progenipoietin (pro-GP) (Fleming et al., 2001, supra; Pulendran et al., 2000, supra; Robinson et al., 2000, supra). Administration of such cytokines for several days to a human or other mammal would enable much larger numbers of precursors to be derived from peripheral blood or bone marrow for in vitro manipulation. Dendritic cells can also be generated from peripheral blood neutrophil precursors in the presence of GM-CSF, IL-4 and TNFα (Kelly et al., 2001, Cell Mol Biol. (Noisy-le-grand) 47, 43-54; Oehler et al., 1998, J Exp Med. 187, 1019-1028). It should be noted that dendritic cells can also be generated, using similar methods, from acute myeloid leukemia cells (Oehler et al., 2000, Ann Hematol. 79, 355-362).

Tissue Dendritic Cell Precursors and Other Sources of APC Precursors:

Other methods for dendritic cell generation exist from, for example, thymic precursors in the presence of IL-3+/−GM-CSF, and liver dendritic cell precursors in the presence of GM-CSF and a collagen matrix. Transformed or immortalized dendritic cell lines may be produced using oncogenes such as v-myc as for example described by (Paglia et al., 1993, J Exp Med. 178(6):1893-1901) or by myb (Banyer and Hapel, 1999, J Leukoc Biol. 66(2):217-223; Gonda et al., 1993, Blood. 82(9):2813-2822).

Circulating Dendritic Cell Precursors:

These have been described in human and mouse peripheral blood. One can also take advantage of particular cell surface markers for identifying suitable dendritic cell precursors. Specifically, various populations of dendritic cell precursors can be identified in blood by the expression of CD11c and the absence or low expression of CD14, CD19, CD56 and CD3 (O'Doherty et al., 1994, Immunology 82, 487-493; O'Doherty et al., 1993, J Exp Med. 178, 1067-1078). These cells can also be identified by the cell surface markers CD13 and CD33 (Thomas et al., 1993b, J Immunol. 151(12), 6840-6852). A second subset, which lacks CD14, CD19, CD56 and CD3, known as plasmacytoid dendritic cell precursors, does not express CD11c, but does express CD123 (IL-3R chain) and HLA-DR (Farkas et al., 2001, Am J Pathol. 159, 237-243; Grouard et al., 1997, J Exp Med. 185, 1101-1111; Rissoan et al., 1999, Science 283, 1183-1186). Most circulating CD11c⁺ dendritic cell precursors are HLA-DR⁺, however some precursors may be HLA-DR⁻. The lack of MHC class II expression has been clearly demonstrated for peripheral blood dendritic cell precursors (del Hoyo et al., 2002, Nature 415, 1043-1047).

Optionally, CD33⁺CD14^{-/lo} or CD11c⁺HLA-DR⁺, lineage marker-negative dendritic cell precursors described above can be differentiated into more mature APCs by incubation for 18-36 hours in culture medium or in monocyte conditioned medium (Thomas et al., 1993b, supra; Thomas and Lipsky, 1994, J Immunol. 153, 4016-4028; O'Doherty et al., 1993, supra). Alternatively, following incubation of peripheral blood non-T cells or unpurified PBMC, the mature peripheral blood dendritic cells are characterized by low density and so can be purified on density gradients, including metrizamide and Nycodenz (Freudenthal and Steinman, 1990, Proc Natl Acad Sci USA 87, 7698-7702; Vremec and Shortman, 1997, J Immunol. 159, 565-573), or by specific monoclonal antibodies, such as but not limited to the CMRF-44 mAb (Fearnley et al., 1999, Blood 93, 728-736; Vuckovic et al., 1998, Exp Hematol. 26, 1255-1264). Plasmacytoid dendritic cells can be purified directly from peripheral blood on the basis of cell surface markers, and then incubated in the presence of IL-3 (Grouard et al., 1997, supra; Rissoan et al., 1999, supra). Alternatively, plasmacytoid DC can be derived from density gradients or CMRF-44 selection of incubated peripheral blood cells as above.

In general, for dendritic cells generated from any precursor, when incubated in the presence of activation factors such as monocyte-derived cytokines, lipopolysaccharide and DNA containing CpG repeats, cytokines such as TNF-α, IL-6, IFN-α, IL-1β, necrotic cells, re-adherence, whole bacteria, membrane components, RNA or polyIC, immature dendritic cells will become activated (Clark, 2002, J Leukoc Biol. 71, 388-400; Hacker et al., 2002, Immunology 105, 245-251; Kaisho and Akira, 2002, Biochim Biophys Acta 1589, 1-13; Koski et al., 2001, Crit Rev Immunol. 21, 179-1890. This process of dendritic cell activation is inhibited in the presence of NF-κB inhibitors (O'Sullivan and Thomas, 2002, J Immunol. 168, 5491-5498).

4.1.2 Effector Cells

The screening methods of the present invention generally include an effector cell, such as a T-cell clone or line, which is capable of specifically recognizing the target antigen that is presented by an MHC molecule. Several methods for generating T-cell lines and clones are known in the art.

Cytotoxic T-cells for use in methods of the invention may be obtained from virtually any source containing T-cells, including but not limited to, peripheral blood (e.g., a peripheral blood mononuclear cell (PBMC) preparation), dissociated organs or tissue, including tumors, synovial fluid (e.g., from arthritic joints), ascites fluid or pleural effusion from cancer patients, cerebral spinal fluid, and the like. Sources of particular interest include tissues affected by diseases, such as cancers, autoimmune diseases, viral infections, bacterial infections, etc. In specific embodiments, cytotoxic T-cells used are used in methods of the invention, and are provided as a clonal population or a near clonal population. Such populations may be produced using conventional techniques, for example, sorting by FACS into individual wells of a microtiter plate, cloning by limited dilution, and the like, followed by growth and replication. In vitro expansion of the desired T lymphocytes can be carried out in accordance with known techniques (including but not limited to those described in U.S. Pat. No. 6,040,177 to Riddell et al.), or variations thereof that will be apparent to those skilled in the art.

Preparatory experiments with the T-cell clones preferably may be carried out to establish the minimal concentration of native peptide ligand which causes sufficient lysis of the HLA-matched APC. A T-cell clone that recognizes the peptide presented by APC such as lymphocytic cell lines (LCL) advantageously may be used to conduct titration experiments using a limiting number of APC which cause optimal recognition by the native peptide at a given concentration.

4.1.3 D-Amino Acid Recognition

The peptide mixtures within each set of the CPL are suitably screened to determine which D-amino acids at each position of the peptide are recognized by a TCR that is known to specifically recognize (i.e., bind to) the target antigen (e.g., matrix protein 1 influenza antigen). The assessment of D-amino acid recognition can suitably be performed by any means known in the art. By way of a non-limiting illustrative example, measurement of target cell (e.g., APC) cytotoxicity can be used. Once the recognized D-amino acids for each position of the peptide are determined, this information can be used to identify individual peptides to be synthesized.

For example, in specific embodiments the best recognized D-amino acid is selected at every position of the peptide. In other embodiments, where one or more index position(s) appears to accommodate (i.e., recognize) a variety of D-amino acids, a number of candidate peptides may be synthesized each with different recognized D-amino acids at the one or more position(s). This allows for further testing to be conducted to confirm recognition and determine number of additional characteristics of the identified D-amino acid peptides (e.g., stability, bioavailability, half-life, toxicity, cytotoxicity, etc.) in order to determine the most favorable D-amino acid peptide.

The selection criteria for selecting the recognized D-amino acids at each position is dependent on the assay being used to perform the screening. By way of an example, if a chemokine secretion assay is used to assess which D-amino acids are recognized by a TCR then the D-amino acid that results in the production of the highest amount or level of the chemokine is selected for inclusion in the identified D-amino acid peptide (as this D-amino acid is the “best recognized” D-amino acid at that index position). At some index positions (“degenerate index positions”), more than one D-amino acid is recognized by the TCR. In these scenarios any of the recognized D-amino acids may be selected for inclusion in the identified D-amino acid peptide. Alternatively, several identified D-amino acid peptides may be produced, with each peptide having different recognized D-amino acid peptide at the degenerate index position. In specific embodiments, a D-amino acid may be selected for inclusion in an identified peptide if the screening assay output (e.g., MIP-1β production) is within about 30%, 25%, 20%, 15%, 12.5%, 10%, 9%, 8%_{, 7}%, 6%, 5%, 4%, 3% or 1% of the functional activity of the best recognized D-amino acid at that index position. In these embodiments, any D-amino acids falling within this range can be selected for inclusion in a D-amino acid peptide upon which further testing of T-cell agonist activity should be conducted.

Recognition of a particular D-amino acid peptide or mixture of peptides may suitably be determined by assessing whether the peptides elicit a cellular immune response directed by virus specific cytotoxic T lymphocytes. Such assays are well known in the art and described in detail, below.

The ability of effector cells (e.g., CTLs) to recognize a specific D-amino acid at an index position of a peptide can be measured by assessing the corresponding peptide mixture as a whole using an ELISPOT assay, chromium release assay, intracellular cytokine staining, or any other method for assessing immunogenicity. When a peptide mixture is identified as being recognized by the effector cell, the D-amino acid at that index position can be identified. This process can be repeated for every peptide set of the CPL, such that information is available for every D-amino acid at each index position.

4.2 Cytotoxicity Assays

When a TCR successfully recognizes an MHC-peptide complex, it is stimulated. This stimulation can be monitored by proliferation of the T-cells (for example, by incorporation of ³H) and/or by production of cytokines and/or chemokines by the T-cells (for example, by an ELISPOT assay). Thus, it is possible to detect the recognition of a CPL D-amino acid peptide by using appropriate target (e.g., APC) and effector (e.g., T-cell) lines.

Any cytotoxicity assay is suitable for using to assess the ability of D-amino acid peptides identified by the screening methods described above and elsewhere herein to elicit a T-cell response. Examples of assays suitable for this purpose are detection of cytokine production or secretion by target antigen-specific T-cells; detection of the surface molecule expression on target antigen-specific T-cells; detection of target cell (e.g., APC) lysis; and/or detection of cell proliferation. Examples of methods suitable for this are a cytokine assay (see, Chapter 6.2 to 6.24 in Current Protocols in Immunology (1999), edited by Coligan J. E., Kruisbeek A. M., Margulies D. H., Shevach E. M. and Strober W., John Wiley & Sons), ELISPOT (see, Chapter 6.19 in Current Protocols in Immunology, supra), a ⁵¹Cr release assay (see, Chapter 3.11 in Current Protocols in Immunology, supra) or detection of proliferation (see, Chapter 3.12 in Current Protocols in Immunology, supra).

In another embodiment, binding of MHC complexes presenting the D-amino acid peptide antigen to the surface of a T-cell (i.e., to a TCR) is detected. This may be carried out such that the MHC complexes are labeled themselves, for example fluorescently labeled, or that, in a further step, an MHC-specific, labeled, for example fluorescently labeled, antibody is used in order to detect the MHC complexes. The fluorescent label of the T-cells can then be measured and evaluated, for example, in a fluorescence-activated cell sorter (FACS). Another possible way of detecting binding of the complexes to the T-cells is again measuring T-cell activation. Suitable measurements may be any method known for measuring T-cell activity, examples of which include cytokine assays, RIA, ELISA, ELISPOT, ⁵¹Cr release assay, RT-PCR, T-cell proliferation assays and flow cytometry-based assays.

4.2.1 Chromium Release Assay

Traditionally, techniques for measuring CD8⁺ cytotoxic T lymphocytes (CTL) activity relied on lysis of autologous target cells (e.g., APC presenting D-amino acid peptide in the context of an MHC). One example of a suitable and convenient assay of this type for assessing cytotoxicity is a chromium (Cr) release assay. A target cell (e.g., an APC presenting the D-amino acid peptide antigen) is loaded with ⁵¹Cr prior to being exposed to an effector cell (e.g., an antigen-specific T-cell). After labeling with ⁵¹Cr, the target and effector cells are mixed together with culture and incubated for several hours, for example, about 2 hours to about 16 hours, or about 2 to about 8 hours, or about 4 hours. After the incubation, the amount of ⁵¹Cr released from the cells (i.e., into the supernatant) is measured. Typically, ⁵¹Cr is measured using a scintillation counter. However, other methods of detection can be used, including fluorometric assessment of T-lymphocyte antigen-specific lysis (FATAL) assays using intracellular dye, CFSE, labeled targets.

Generally, it is preferred to perform control incubations to test spontaneous Cr release in the absence of effector cells and maximal total release as controls with each screening assay that is performed.

For example, APC described above and with a candidate D-amino acid peptide may be screened for the lysis by a target antigen-specific T-cell. Suitable T-cells may be prepared from PBMC from healthy virus seropositive blood donors. The cells may be depleted of CD4⁺, CD16⁺, and CD56⁺ cells by any method known in the art, such as by immunomagnetic separation using specific antibodies, and the like. Alternatively, CD8⁺ cells may be purified or semi-purified CTL may be kept in culture and used as a source of virus specific T-cell clones. In vitro stimulation of the T-cell cultures with γ-irradiated fresh, allogeneic PBMC and PHD causes proliferation of the specific T-cell clones. A combination of limiting dilution and in vitro stimulation allows the desired clones to be expanded.

The Cr release assay indicates target cell (e.g., APC) membrane disruption resulting from release of performin and granzymes by the effector cell (e.g., antigen-specific T-cell). These assays are commonly performed in the art, especially in vaccine studies.

4.2.2 Cytokine and/or Chemokine Secretion by T-Cells

Other methods for screening immunogenic activity include techniques well known to the skilled artisan (for example, immunoassays such as ELISA, or T-cell stimulation assays). By way of an illustrative example, such screening methods may be performed using methods such as those described in Harlow and Lane, Antibodies: A Laboratory Manual, Cold Spring Harbor Laboratory, 1988. Increased levels of a Th1-type cytokines and/or chemokines (e.g., MIP-1α, MIP-1β, IFN-γ, IL-1, IL-2, IL-8, IL-12, IL-18, TFNβ, CD107a, and RANTES) are clear indicators that a D-amino acid peptide has induced or enhanced a cell-mediated immune responses (and is, therefore, recognized by the target antigen-specific TCR).

Cytotoxicity is typically mediated by CD8⁺ T-cells and characterized by the release of cytokines and/or chemokines including IFN-γ, IL-1, IL-2, IL-8, IL-12, IL-18, and TFNβ; and chemokines including monokine induced by interferon-γ (MIG), interferon-γ-inducible protein-10 (IP-10), monocyte chemoattractant protein-1 (MCP-1), regulated upon activation, normal I-cell expressed and secreted (RANTES), macrophage inflammatory protein (MIP)-1α, and MIP-1β, and CD107a.

Measurement of virus-specific CD8⁺ T-cell activity by techniques such as ELISPOT, MHC-peptide tetramer staining, and flow cytometric analysis of intracellular cytokine and/or chemokine production, offers advantages over the cell lysis assays (e.g., the chromium-release assay described above). For example, improved sensitivity and more-rapid delineation of MHC-I-restricted epitopes, compared with that of Cr release T-cell assays. Also, these assays are typically have simpler methodology, require fewer cells, and obviate the need to expand and clone antigen-specific T lymphocytes and/or establish autologous B lymphoblastoid cell lines.

4.2.3 MHC Multimer Assays

MHC multimer (e.g., tetramer) staining is more sensitive than traditional methods for detecting antigen-specific effector cells (e.g., T-cells). However, the use of this technique is restricted to well known epitopes in association with defined MHC alleles, and requires subsequent culture for functional characterization of tetramer-positive cells.

In some embodiments, MHC tetramers presenting a D-amino acid peptide are generated by biotinylating MHC monomers using methods well known in the art. Tetrameric MHC (“tetramers”) can then be constructed by the addition of PE-conjugated streptavidin at a suitable MHC:streptavidin molar ratio (e.g., 4:1). Effector cells (e.g., T-cells) can be stained with PE-conjugated tetramer, prior to staining with aminoactinomycin D, before analyzing using flow cytometry.

MHC multimers, such as MHC peptide tetramers (as described in European Patent No. EP812331) and MHC peptides pentamers (as described in International PCT Application Publication No. WO2004/018520), have been developed in order to label and detect (e.g., by flow cytometry) T-cells in a sample that react to an MHC binding peptide of interest. Definitive or confirmatory information as to whether a identified D-amino acid peptide is a T-cell agonist by using MHC multimers loaded with the candidate D-amino acid peptide. Although theoretically possible, in practice would be very labour intensive and time consuming to attempt to construct fully purified functional MHC multimers for a large number of peptides, e.g., every peptide in a CPL. As a consequence, MHC multimers have been used primarily in the past only to confirm recognition of D-amino acid peptide by antigen-specific effector cells (e.g., T-cells), once such recognition is identified from the initial CPL screening methods, as described above and elsewhere herein.

4.2.4 Flow-Based Assays

In a flow-based assay (including some of those described above), the sample is detected (e.g., by fluorescence or other means) while flowing through detection windows. Flow-based assays include, for example, flow cytometry. An advantage of a flow cytometry-based assay is that it can be technically simple and a relatively rapid and objective method for identifying antigen-specific cells.

Flow cytometry-based intracellular staining methods provide a technically simple and relatively fast method for identifying antigen-responsive T-cells that recognize candidate D-amino acid peptides present in a CPL. Success with intracellular staining assays have most frequently reported in studies evaluating CD4⁺ T-cell responses to viral antigens using PBMC from chronically infected individuals (for example, CMV or HIV). The use of MHC tetramers (such as those described above) is beneficial for the detection of ex vivo antigen-specific cells in CD4⁺ or CD8⁺ T-cells from immunized individuals, especially in embodiments wherein the number of circulating antigen-specific effector cells (e.g., T-cells) may be considerably lower than that found in individuals exposed to the infectious agent.

4.2.5 ELISA/ELISPOT Assays

Enzyme-linked immunosorbent assay (ELISA) has long been used to detect and measure cytokine and/or chemokine levels. ELISA is commonly used in the evaluation and characterization of immune responses, and methods for performing such analysis are well known in the art. In some embodiments, a multi-analyte ELISA assay may be performed, with allows for the rapid screening of the secretion of up to 12 cytokines and/or chemokines in a single experiment.

Variations of the traditional ELISA method, including ELISPOT assays, are also well known in the art.

The enzyme linked immunospot (ELISPOT) assay (such as described in European Patent No. EP0941478) can measure the activation of immobilized effector cells (e.g., T-cells) in a sample by detecting cytokines secreted by effector cells (e.g., T-cells) in response to stimulation with a recognized D-amino acid peptide through target cells (e.g., APC) through capture of the cytokines and/or chemokines secreted by responding effector cells (e.g., T-cells) in their vicinity on an adsorber membrane with a cytokine- and/or chemokine-specific antibody (e.g., monoclonal antibody). The cytokine capture is then detected with a second anti-cytokine antibody, which binds to a different epitope on the same cytokine compared to the capture antibody. Binding of the second antibody is then detected with a color-based labeling reaction. As a consequence, stimulated cells are then detected as colored spots on the membrane, which form around the original location where the cell was immobilized.

This technique therefore enables the enumeration of lymphocytes secreting a specific cytokine and/or chemokine in response to a recognized D-amino acid peptide. Basically, cytokine and/or chemokine secreting cells (e.g., T-cells, NK cells, etc.) may be revealed by culturing the cells in specially modified ELISA wells that contain antibody to the cytokine and/or chemokine of interest bound to the well surface, together with MHC molecules presenting the candidate D-amino acid peptide (or mixture of candidate D-amino acid peptides). In this method, the standard ELISA reagents are replaced with enzyme-substrate complexes that yield a colored precipitate (spots), adjacent to the secreting cell. Spots can then be counted to give a measure of the number of cytokine- and/or chemokine-producing cells.

Further advantage of performing ELISPOT assays is that advanced operator training and expensive equipment are not necessary for measuring frequency of responses. In some embodiments, ELISPOT evaluates interferon-γ (IFN-γ) production, which occurs in large quantities as this cytokine is considered to be prototypic of a Th1 response. However, other cytokines can be assessed and the measurement of which is suitable for the present invention. ELISPOT analysis may be improved by adding a computer-assisted microscope to simplify the assay readout, and allows batch analysis of large series of samples, and facilitates standardization (as described in Samri et al., 2006).

When the candidate D-amino acid peptide T-cell agonists have been screened, peptide or peptide groups which have resulted in significant immunogenicity may be individually screened in the same manner to confirm activity. Additional assays may be performed with identified peptides if desired. In this regard, the screening of the positional libraries point to several CTL peptide epitope analogues which typically result in an equivalent or increased cytotoxicity than the native peptide by chromium release assay. Sequences corresponding to the best recognized defined amino acid library at each position of the peptide may then be selected, and a number of identified peptides synthesized. At positions where the differences in cytotoxicity are observed to be minimal between D-amino acids, all or several (i.e., 20, 19, 18, 17, 16, 15, 14, 13, 12, 11, 10, 9, 8, 7, 6, 5, 4, 3, 2, or 1) of the recognized D-amino acids are available for selection. Advantageously, several peptides each incorporating a different recognized D-amino acid at certain positions may advance to further screening, if desired, in order to determine which D-amino acid peptides are most immunogenic.

### 5. D-Amino Acid Peptides

5.1 Methods of Producing D-Amino Acid Peptides

Once identified using the methods described above, candidate D-amino acid peptides can be synthesized for therapeutic uses. In some embodiments the D-amino acid peptide antigen can be synthesized using solution synthesis, solid phase synthesis (as described, for example, by Atherton and Sheppard (Solid Phase Peptide Synthesis: A Practical Approach, IRL Press at Oxford University Press, Oxford, England, 1989) or by Roberge et al. (1995, Science 269: 202)), or using bioreactors exploiting organisms with expanded genetic codes (as described in Neuman, (2012, FEBS Lett. 586:2057-2064).

Usually, the D-amino acid peptides are at least 6, 7, 8, 9, 10, 11, 12, 13, 14, 15, 20, 25, 30 D-amino acid residues in length. In some embodiments, the D-amino acid sequence recognized by the target antigen-specific T-cell makes up part of a longer polypeptide sequence.

An advantage of the present invention is the increased ability of D-amino acid peptide epitopes to navigate host barriers associated with route of entry, as compared with their L-amino acid peptide counterparts. Such barriers include serum complement/proteases, gastric acid and digestive enzymes. Thus, the peptides of the present invention are suitably formulated in pharmaceutical compositions to take advantage of the superior stability of D-amino acid peptides in human serum and simulated gastric acid. The enhanced stability of D-amino acid peptides in vivo indicates that the D-amino acid peptides identified using the methods of the present invention are likely to enable a more effective immune priming relative to any counterpart L-amino acid peptide.

5.2 D-Amino Acid Peptides for Treating Influenza

In one aspect the present invention provides D-amino acid peptides that are of particular use in eliciting an immune response against Matrix M1 antigen from influenza. The nature of the positional scanning method CPL described above allows for detailed information regarding D-amino degeneracy at particular index positions.

For example, a target-antigen specific TCR may recognize only a single D-amino acid at some index positions, whereas the TCR may at other index positions recognize a plurality of D-amino acids. By way of an illustrative example, the present inventors found that the ALF3 TCR clone, that is specific for the well characterized GILGFVFTL₅₈₋₆₆ peptide epitope of the influenza matrix M1 protein recognized two D-amino acids at positions 1, 2, 4 and 5 of a nonamer, but only a single D-amino acid at positions 3, 6, 7, 8, and 9. Accordingly, the present invention provides an immunogenic D-amino acid peptide that comprises, consists of, or consists essentially of the amino acid sequence represented by formula I:

X₁X₂pX₃X₄nnpp (I)

wherein:
X₁ is selected from g or r;
X₂ is selected from p or f;
X₃ is selected from p or q; and
X₄ is selected from w or g.

In specific embodiments, suitable D-amino acid sequences in accordance with Formula I include those with D-amino acid sequences as set forth in SEQ ID NO: 3-10. In even more specific illustrative embodiments, the D-amino acid sequence set forth in SEQ ID NO:3 is suitable for eliciting both mucosal and systemic immunity to the matrix M1 protein from influenza. Accordingly, D-amino acid peptides comprising, consisting or consisting essentially of a D-amino acid sequence in accordance with formula I (and therefore including any one of the sequences set forth in SEQ ID NO:3-10) are particularly suitable for formulating in pharmaceutical compositions for eliciting an immune response against the influenza virus, as described in further detail below. These sequences are extremely biologically stable, and can prime influenza-specific T-cells as efficiency as the native L-amino acid peptide. Furthermore, the T-cell populations primed by the D-amino acid peptides identified by the methods disclosed herein can suitably be characterized as (i) exhibiting natural polyfunctional profiles; (ii) encompass TCR repertoires that closely mimic the target antigen; and (iii) exhibit the ability to recognize endogenously presented matrix M1 antigen from influenza.

### 6. Pharmaceutical Formulations

In accordance with the present invention, the D-amino acid peptides identified using the CPL and methods described above, are useful in compositions and methods for enhancing or eliciting an immune response to a target antigen (e.g., a viral antigen). These compositions are useful, therefore, for treating a disease or condition that is associated with the target antigen (e.g., in which an effective cytotoxic immune response against the target antigen is required).

Suitable diseases suitable for treatment with a D-amino acid peptide of the invention, include infection with a virus, bacteria, fungi, or parasite. Viruses include, but are not limited to: Orthomyxoviridae (e.g., influenza viruses, including swine influenza); Retroviridae human immunodeficiency viruses, such as HIV-1 (also referred to as HTLV-III, LAV or HTLV-III/LAV, or HIV-III); and other isolates, such as HIV-LP); Picornaviridae (e.g., polio viruses, hepatitis A virus; enteroviruses, human Coxsackie viruses, rhinoviruses, echoviruses); Calciviridae (e.g., strains that cause gastroenteritis, including Norwalk and related viruses); Togaviridae (e.g., equine encephalitis viruses, rubella viruses); Flaviridae (e.g., dengue viruses, encephalitis viruses, yellow fever viruses; Zika virus); Coronoviridae (e.g., coronaviruses); Rhabdoviradae (e.g., vesicular stomatitis viruses, rabies viruses); Filoviridae (e.g., ebolaviruses); Paramyxoviridae (e.g., parainfluenza viruses, mumps virus, measles virus, respiratory syncytial virus, metaneumovirus); Bungaviridae (e.g., Hantaan viruses, bunga viruses, phleboviruses and Nairo viruses); Arenaviridae (hemorrhagic fever viruses); Reoviridae (e.g., reoviruses, orbiviurses and rotaviruses); Bimaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus); Poxyiridae (variola viruses, VACV, pox viruses); and Iridoviridae (e.g., African swine fever virus); and unclassified viruses (e.g., the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted (i.e., Hepatitis C); and astroviruses.

In some embodiments, the pathogenic infection is a bacterial pathogen. Bacteria from which pathogenic infection is known to occur in a subject include, but are not limited to, pathogenic Pasteurella species (e.g., Pasteurella multocida), Staphylococci species (e.g., Staphylococcus aureus), Streptococcus species (e.g., Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae), Neisseria species (e.g., Neisseria gonorrhoeae, Neisseria meningitidis), Escherichia species (e.g., enterotoxigenic E. coli (ETEC), enteropathogenic E. coli (EPEC), enterohemorrhagic E. coli (EHEC), and enteroinvasive E. coli (ETEC)), Bordetella species, Campylobacter species, Legionella species (e.g., Legionella pneumophila), Pseudomonas species, Shigella species, Vibrio species, Yersinia species, Salmonella species, Haemophilus species (e.g., Haemophilus influenzae), Brucella species, Francisella species, Bacterioides species, Clostridia species (e.g., Clostridium difficile, Clostridium perfringens, Clostridium tetani), Mycobacteria species (e.g., M. tuberculosis, M. avium, M. intracellulare, M. kansaii, M. gordonae), Helicobacter pyloris, Borelia burgdorferi, Listeria monocytogenes, Chlamydla trachomatis, Enterococcus species, Bacillus anthracis, Corynebacterium diphtheriae, Erysipelothrix rhusiopathiae, Enterobacter aerogenes, Klebslella pneumonlae, Fusobacterium nucleatum, Streptobadllus monllbformis, Treponema pallldlum, Treponema pertenue, Leptospira, Rickettsia, and Actinomyces israeli.

In other embodiments of the invention, the pathogenic infection is a eukaryotic pathogen, such as pathogenic fungi and parasites. Fungi that are known to be pathogenic at least to some extent include, but are not limited to, Cryptococcus neoformans, Histoplasma capsulatum, Coccidioides immitis, Blastomyces dermatitidis, Candida albicans, Candida glabrata, Aspergillus fumigata, Aspergillus flavus, and Sporothrix schenckii.

Other eukaryotic pathogens from which the heterologous antigen can be derived include, but are not limited to, pathogenic protozoa, helminths, Plasmodium, such as Plasmodium falciparum, Plasmodium malariae, Plasmodium ovale, and Plasmodium vivax; Toxoplasma gondii; Trypanosoma brucei, Trypanosoma cruzi; Schistosoma haematobium, Schistosoma mansoni, Schistosoma japonicum; Leishmania donovani; Giardia intestinalis; Cryptosporidium parvum; and the like.

Other diseases and/or conditions that the present invention can be used to treat include malignant or pre-malignant conditions, proliferative or hyper-proliferative conditions, or any disease arising or deriving from or associated with a functional or other disturbance or abnormality in the proliferative capacity or behavior of any cells or tissues of the body. Thus, the methods described herein could be used to diagnose a cancer, including assessing the likelihood whether a cancer is a metastatic cancer. For example, cancers which could be suitably diagnosed in accordance with the practices of this invention include breast cancer, colon cancer, lung cancer and prostate cancer, cancers of the blood and lymphatic systems (including Hodgkin's disease, leukemias, lymphomas, multiple myeloma, and Waldenstrom's disease), skin cancers (including malignant melanoma), cancers of the digestive tract (including head and neck cancers, esophageal cancer, stomach cancer, cancer of the pancreas, liver cancer, colon and rectal cancer, anal cancer), cancers of the genital and urinary systems (including kidney cancer, bladder cancer, testis cancer, prostate cancer), cancers in women (including breast cancer, ovarian cancer, gynecological cancers and choriocarcinoma) as well as in brain, bone carcinoid, nasopharyngeal, retroperitoneal, thyroid and soft tissue tumors.

The compositions of the present invention are, therefore, useful for treating a disease or condition that is associated with the target antigen (e.g., by enhancing or eliciting an immune response against a target antigen). The pharmaceutical composition may comprise a pharmaceutically acceptable carrier, excipient, or diluent. In some embodiments, the compositions are administered to individuals having the disease and/or condition associated with the target antigen. In other embodiments, the compositions are administered to at-risk individuals who are at risk of developing the disease or condition associated with the target antigen.

Pharmaceutical compositions suitable for use in the present invention include compositions wherein the D-amino acid peptide antigen is contained in an effective amount to achieve their intended purpose. The dose of active compound(s) administered to a patient should be sufficient to achieve a beneficial response in the patient over time such as a reduction in at least one symptom associated with the disease and/or condition, which is suitably associated with a condition selected from pathogenic infection or cancer. The quantity or dose frequency of the pharmaceutically active compounds(s) to be administered may depend on the subject to be treated inclusive of the age, sex, weight and general health condition thereof. In this regard, precise amounts of the active compound(s) for administration will depend on the judgement of the practitioner. In determining the effective amount of the active compound(s) to be administered in the treatment of the disease or condition, the practitioner may evaluate inflammation, pro-inflammatory cytokine levels, lymphocyte proliferation, cytolytic T lymphocyte activity and regulatory T lymphocyte function. In any event, those of skill in the art may readily determine suitable dosages of the D-amino acid peptide.

Accordingly, the bioactive agents are administered to a subject to be treated in a manner compatible with the dosage formulation, and in an amount that will be prophylactically and/or therapeutically effective. The amount of the composition to be delivered, generally in the range of from 0.01 μg/kg to 100 μg/kg of D-amino acid peptide per dose, depends on the subject to be treated. In some embodiments, and dependent on the intended mode of administration, the D-amino acid peptide containing compositions will generally contain about 0.1% to 90%, about 0.5% to 50%, or about 1% to about 25%, by weight peptide, the remainder being suitable pharmaceutical carriers and/or diluents etc.

Depending on the specific condition being treated, the pharmaceutical compositions may be formulated and administered systemically, topically or locally. Techniques for formulation and administration may be found in “Remington's Pharmaceutical Sciences”, Mack Publishing Co., Easton, Pa., latest edition. Suitable routes may, for example, include oral, rectal, transmucosal, or intestinal administration; parenteral delivery, including intramuscular, subcutaneous, transcutaneous, intradermal, intramedullary delivery (e.g., injection), as well as intrathecal, direct intraventricular, intravenous, intraperitoneal, intranasal, or intraocular delivery (e.g., injection). For injection, the bioactive agents of the invention may be formulated in aqueous solutions, suitably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

The compositions of the present invention may be formulated for administration in the form of liquids, containing acceptable diluents (such as saline and sterile water), or may be in the form of lotions, creams or gels containing acceptable diluents or carriers to impart the desired texture, consistency, viscosity and appearance. Acceptable diluents and carriers are familiar to those skilled in the art and include, but are not restricted to, ethoxylated and nonethoxylated surfactants, fatty alcohols, fatty acids, hydrocarbon oils (such as palm oil, coconut oil, and mineral oil), cocoa butter waxes, silicon oils, pH balancers, cellulose derivatives, emulsifying agents such as non-ionic organic and inorganic bases, preserving agents, wax esters, steroid alcohols, triglyceride esters, phospholipids such as lecithin and cephalin, polyhydric alcohol esters, fatty alcohol esters, hydrophilic lanolin derivatives, and hydrophilic beeswax derivatives.

Alternatively, the bioactive agents of the present invention can be formulated readily using pharmaceutically acceptable carriers well known in the art into dosages suitable for oral administration, which is also contemplated for the practice of the present invention. Such carriers enable the bioactive agents of the invention to be formulated in dosage forms such as tablets, pills, capsules, liquids, gels, syrups, slurries, suspensions and the like, for oral ingestion by a patient to be treated. These carriers may be selected from sugars, starches, cellulose and its derivatives, malt, gelatin, talc, calcium sulfate, vegetable oils, synthetic oils, polyols, alginic acid, phosphate buffered solutions, emulsifiers, isotonic saline, and pyrogen-free water.

Pharmaceutical formulations for parenteral administration include aqueous solutions of the particles in water-soluble form. Additionally, suspensions of the bioactive agents may be prepared as appropriate oily injection suspensions. Suitable lipophilic solvents or vehicles include fatty oils such as sesame oil, or synthetic fatty acid esters, such as ethyl oleate or triglycerides. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions.

Pharmaceutical preparations for oral use can be obtained by combining the D-amino acid peptides with solid excipients and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Such compositions may be prepared by any of the methods of pharmacy but all methods include the step of bringing into association one or more therapeutic agents as described above with the carrier which constitutes one or more necessary ingredients. In general, the pharmaceutical compositions of the present invention may be manufactured in a manner that is itself known, e.g., by means of conventional mixing, dissolving, granulating, dragee-making, levigating, emulsifying, encapsulating, entrapping or lyophilizing processes.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of particle doses.

Pharmaceuticals which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

The D-amino acid peptides of the present invention may be administered over a period of hours, days, weeks, or months, depending on several factors, including the severity of the condition being treated, whether a recurrence of the condition is considered likely, etc. The administration may be constant, e.g., constant infusion over a period of hours, days, weeks, months, etc. Alternatively, the administration may be intermittent, e.g., D-amino acid peptides may be administered once a day over a period of days, once an hour over a period of hours, or any other such schedule as deemed suitable.

The D-amino acid peptides of the present invention may also be administered to the respiratory tract as a nasal or pulmonary inhalation aerosol or solution for a nebulizer, or as a microfine powder for insufflation, alone or in combination with an inert carrier such as lactose, or with other pharmaceutically acceptable excipients. In some particulate embodiments of the present invention, the particles of a formulation may advantageously have diameters of less than 50 μm, suitably less than 10 μm.

In some embodiments, the pharmaceutical formulation comprising the D-amino acid peptides of the invention could be administered as a cellular preparation. When APC or regulatory lymphocytes are employed, the cells can be introduced into a patient by any means (e.g., injection), which produces the desired modified immune response to an antigen or group of antigens. The cells may be derived from the patient (i.e., autologous cells) or from an individual or individuals who are MHC-matched or -mismatched (i.e., allogeneic) with the patient. In specific embodiments, autologous cells are injected back into the patient from whom the source cells were obtained. The injection site may be subcutaneous, intraperitoneal, intramuscular, intradermal, or intravenous. The cells may be administered to a patient already suffering from the unwanted immune response or who is predisposed to the unwanted immune response in sufficient number to prevent or at least partially arrest the development, or to reduce or eliminate the onset of, that response. The number of cells injected into the patient in need of the treatment or prophylaxis may vary depending on inter alia, the antigen or antigens and size of the individual. This number may range for example between about 10³ and 10¹¹, and usually between about 10⁵ and 10⁷ cells (e.g., macrophages, dendritic cells etc. or their precursors). Single or multiple administrations of the cells can be carried out with cell numbers and pattern being selected by the treating physician. The cells should be administered in a pharmaceutically acceptable carrier, which is non-toxic to the cells and the individual. Such carrier may be the growth medium in which the cells were grown, or any suitable buffering medium such as phosphate buffered saline. The cells may be administered alone or as an adjunct therapy in conjunction with other therapeutics known in the art for the treatment of diseases or conditions associated with the target antigen, and/or other forms of specific immunotherapy. In specific embodiments, the APC are pre-contacted with the D-amino acid peptide or are administered concurrently to the subject with one or more such peptide antigens.

6.1 Particulate Compositions

In some embodiments, the D-amino acid peptides of the invention are formulated in particulate form. A variety of particles may be used in the invention, including but not limited to, liposomes, micelles, lipidic particles, ceramic/inorganic particles and polymeric particles, and are typically selected from nanoparticles and microparticles. The particles are suitably sized for phagocytosis or endocytosis by APCs.

In some embodiments, the particles comprise an antigen-binding molecule on their surface, which is immuno-interactive with a marker that is expressed at higher levels on APCs (e.g., dendritic cells) than on non-APCs. Illustrative markers of this type include MGL, DCL-1, DEC-205, macrophage mannose R, DC-SIGN or other DC or myeloid specific (lectin) receptors, as for example disclosed by Hawiger et al. (2001, J Exp Med 194, 769), Kato et al. 2003, J Biol Chem 278, 34035), Benito et al. (2004, J Am Chem Soc 126, 10355), Schjetne, et al. (2002, Int Immunol 14, 1423) and van Vliet et al., 2006, Nat Immunol September 24; [Epub ahead of print])(van Vliet et al., Immunobiology 2006, 211:577-585).

The particles can be prepared from a combination of D-amino acid peptides, and a surfactant, excipient or polymeric material. In some embodiments, the particles are biodegradable and biocompatible, and optionally are capable of biodegrading at a controlled rate for delivery of a therapeutic or diagnostic agent. The particles can be made of a variety of materials. Both inorganic and organic materials can be used. Polymeric and non-polymeric materials, such as fatty acids, may be used. Other suitable materials include, but are not limited to, gelatin, polyethylene glycol, trehalulose, dextran and chitosan. Particles with degradation and release times ranging from seconds to months can be designed and fabricated, based on factors such as the particle material.

6.1.1 Polymeric Particles

In some particulate embodiments, the D-amino acid peptides are administered for active uptake by cells, for example by phagocytosis, as described for example in U.S. Pat. No. 5,783,567 (Pangaea). In some embodiments, phagocytosis by these cells may be improved by maintaining a particle size typically below about 20 μm, and preferably below about 11 μm.

In specific particulate embodiments, D-amino acid peptides in particulate form are delivered directly into the bloodstream (i.e., by intravenous or intra-arterial injection or infusion) if uptake by the phagocytic cells of the reticuloendothelial system (RES), including liver and spleen, is desired. Alternatively, one can target, via subcutaneous injection, take-up by the phagocytic cells of the draining lymph nodes. The particles can also be introduced intradermally (i.e., to the APCs of the skin, such as dendritic cells and Langerhans cells) for example using ballistic or microneedle delivery. Illustrative particle-mediated delivery techniques include explosive, electric or gaseous discharge delivery to propel carrier particles toward target cells as described, for example, in U.S. Pat. Nos. 4,945,050, 5,120,657, 5,149,655 and 5,630,796. Non-limiting examples of microneedle delivery are disclosed in International Publication Nos. WO 2005/069736 and WO 2005/072630 and U.S. Pat. Nos. 6,503,231 and 5,457,041.

In other specific particulate embodiments, the route of particle delivery is via the gastrointestinal tract, e.g., orally. Alternatively, the particles can be introduced into organs such as the lung (e.g., by inhalation of powdered microparticles or of a nebulized or aerosolized solution containing the microparticles), where the particles are picked up by the alveolar macrophages, or may be administered intranasally or buccally. Once a phagocytic cell phagocytoses the particle, the D-amino acid peptide is released into the interior of the cell.

Polymeric particles may be formed from any biocompatible and desirably biodegradable polymer, copolymer, or blend. The polymers may be tailored to optimize different characteristics of the particle including: i) interactions between the bioactive agents to be delivered and the polymer to provide stabilization of the bioactive agents and retention of activity upon delivery; ii) rate of polymer degradation and, thereby, rate of agent release profiles; iii) surface characteristics and targeting capabilities via chemical modification; and iv) particle porosity.

Surface eroding polymers such as polyanhydrides may be used to form the particles. For example, polyanhydrides such as poly[(p-carboxyphenoxy)-hexane anhydride] (PCPH) may be used. Biodegradable polyanhydrides are described in U.S. Pat. No. 4,857,311.

In other embodiments, bulk eroding polymers such as those based on polyesters including poly(hydroxy acids) or poly(esters) can be used. For example, polyglycolic acid (PGA), polylactic acid (PLA), or copolymers thereof may be used to form the particles. The polyester may also have a charged or functionalizable group, such as an amino acid. In illustrative examples, particles with controlled release properties can be formed of poly(D,L-lactic acid) and/or poly(D,L-lactic-co-glycolic acid) (“PLGA”) which incorporate a surfactant such as DPPC.

Other polymers include poly(alkylcyanoacrylates), polyamides, polycarbonates, polyalkylenes such as polyethylene, polypropylene, poly(ethylene glycol), poly(ethylene oxide), poly(ethylene terephthalate), poly vinyl compounds such as polyvinyl alcohols, polyvinyl ethers, and polyvinyl esters, polymers of acrylic and methacrylic acids, celluloses and other polysaccharides, and peptides or proteins, or copolymers or blends thereof. Polymers may be selected with or modified to have the appropriate stability and degradation rates in vivo for different controlled drug delivery applications.

In some embodiments, particles are formed from functionalized polyester graft copolymers, as described in Hrkach et al. (1995, Macromolecules 28:4736-4739; and “Poly(L-Lactic acid-co-amino acid) Graft Copolymers: A Class of Functional Biodegradable Biomaterials” in Hydrogels and Biodegradable Polymers for Bioapplications, ACS Symposium Series No. 627, Raphael M. Ottenbrite et al., Eds., American Chemical Society, Chapter 8, pp. 93-101, 1996.)

Materials other than biodegradable polymers may be used to form the particles. Suitable materials include various non-biodegradable polymers and various excipients. The particles also may be formed of the bioactive agent(s) and surfactant alone.

Polymeric particles may be prepared using single and double emulsion solvent evaporation, spray drying, solvent extraction, solvent evaporation, phase separation, simple and complex coacervation, interfacial polymerization, and other methods well known to those of ordinary skill in the art. Particles may be made using methods for making microspheres or microcapsules known in the art, provided that the conditions are optimized for forming particles with the desired diameter.

Methods developed for making microspheres for delivery of encapsulated agents are described in the literature, for example, as described in Doubrow, M., Ed., “Microcapsules and Nanoparticles in Medicine and Pharmacy”, CRC Press, Boca Raton, 1992. Methods also are described in Mathiowitz and Langer (1987, J. Controlled Release 5, 13-22); Mathiowitz et al. (1987, Reactive Polymers 6, 275-283); and Mathiowitz et al. (1988, J. Appl. Polymer Sci. 35, 755-774) as well as in U.S. Pat. Nos. 5,213,812, 5,417,986, 5,360,610, and 5,384,133. The selection of the method depends on the polymer selection, the size, external morphology, and crystallinity that is desired, as described, for example, by Mathiowitz et al. (1990, Scanning Microscopy 4: 329-340; 1992, J. Appl. Polymer Sci. 45, 125-134); and Benita et al. (1984, J. Pharm. Sci. 73, 1721-1724).

In solvent evaporation, described for example, in Mathiowitz et al., (1990), Benita; and U.S. Pat. No. 4,272,398 to Jaffe, the polymer is dissolved in a volatile organic solvent, such as methylene chloride. Several different polymer concentrations can be used, for example, between 0.05 and 2.0 g/mL. The bioactive agent(s), either in soluble form or dispersed as fine particles, is (are) added to the polymer solution, and the mixture is suspended in an aqueous phase that contains a surface-active agent such as poly(vinyl alcohol). The aqueous phase may be, for example, a concentration of 1% poly(vinyl alcohol) w/v in distilled water. The resulting emulsion is stirred until most of the organic solvent evaporates, leaving solid microspheres, which may be washed with water and dried overnight in a lyophilizer. Microspheres with different sizes (between 1 and 1000 μm) and morphologies can be obtained by this method.

Solvent removal was primarily designed for use with less stable polymers, such as the polyanhydrides. In this method, the agent is dispersed or dissolved in a solution of a selected polymer in a volatile organic solvent like methylene chloride. The mixture is then suspended in oil, such as silicon oil, by stirring, to form an emulsion. Within 24 hours, the solvent diffuses into the oil phase and the emulsion droplets harden into solid polymer microspheres. Unlike the hot-melt microencapsulation method described for example in Mathiowitz et al. (1987, Reactive Polymers 6:275), this method can be used to make microspheres from polymers with high melting points and a wide range of molecular weights. Microspheres having a diameter for example between one and 300 microns can be obtained with this procedure.

With some polymeric systems, polymeric particles prepared using a single or double emulsion technique, vary in size depending on the size of the droplets. If droplets in water-in-oil emulsions are not of a suitably small size to form particles with the desired size range, smaller droplets can be prepared, for example, by sonication or homogenation of the emulsion, or by the addition of surfactants.

If the particles prepared by any of the above methods have a size range outside of the desired range, particles can be sized, for example, using a sieve, and further separated according to density using techniques known to those of skill in the art.

The polymeric particles can be prepared by spray drying. Methods of spray drying, such as that disclosed in PCT WO 96/09814 by Sutton and Johnson, disclose the preparation of smooth, spherical microparticles of a water-soluble material with at least 90% of the particles possessing a mean size between 1 and 10 μm.

6.1.2 Ceramic Particles

Ceramic particles may also be used to deliver the bioactive agents of the invention. These particles are typically prepared using processes similar to the well known sol-gel process and usually require simple and room temperature conditions as described for example in Brinker et al. (“Sol-Gel Science: The Physics and Chemistry of Sol-Gel Processing;” Academic Press: San Diego, 1990, p-60), and Avnir et al. (1994, Chem. Mater. 6, 1605). Ceramic particles can be prepared with desired size, shape and porosity, and are extremely stable. These particles also effectively protect doped molecules (polypeptides, drugs etc.) against denaturation induced by extreme pH and temperature (Jain et al., 1998, J. Am. Chem. Soc. 120, 11092-11095). In addition, their surfaces can be easily functionalized with different groups (Lal et al., 2000, Chem. Mater. 12, 2632-2639; Badley et al., 1990, Langmuir 6, 792-801), and therefore they can be attached to a variety of monoclonal antibodies and other ligands in order to target them to desired sites in vivo.

Various ceramic particles have been described for delivery in vivo of active agent-containing payloads. For example, British Patent 1 590 574 discloses incorporation of biologically active components in a sol-gel matrix. International Publication WO 97/45367 discloses controllably dissolvable silica xerogels prepared via a sol-gel process, into which a biologically active agent is incorporated by impregnation into pre-sintered particles (1 to 500 μm) or disks. International Publication WO 0050349 discloses controllably biodegradable silica fibres prepared via a sol-gel process, into which a biologically active agent is incorporated during synthesis of the fibre. U.S. Pat. Appl. Pub. 20040180096 describes ceramic nanoparticles in which a bioactive substance is entrapped. The ceramic nanoparticles are made by formation of a micellar composition of the dye. The ceramic material is added to the micellar composition and the ceramic nanoparticles are precipitated by alkaline hydrolysis. U.S. Pat. Appl. Pub. 20050123611 discloses controlled release ceramic particles comprising an active material substantially homogeneously dispersed throughout the particles. These particles are prepared by mixing a surfactant with an apolar solvent to prepare a reverse micelle solution; (b) dissolving a gel precursor, a catalyst, a condensing agent and a soluble active material in a polar solvent to prepare a precursor solution; (c) combining the reverse micelle solution and the precursor solution to provide an emulsion and (d) condensing the precursor in the emulsion. U.S. Pat. Appl. Pub. 20060210634 discloses adsorbing bioactive substances onto ceramic particles comprising a metal oxide (e.g., titanium oxide, zirconium oxide, scandium oxide, cerium oxide and yttrium oxide) by evaporation. Kortesuo et al. (2000, Int J Pharm. May 10; 200(2):223-229) disclose a spray drying method to produce spherical silica gel particles with a narrow particle size range for controlled delivery of drugs such as toremifene citrate and dexmedetomidine HCl. Wang et al. (2006, Int J Pharm. 308(1-2):160-167) describe the combination of adsorption by porous CaCO3 microparticles and encapsulation by polyelectrolyte multilayer films for delivery of bioactive substances.

6.1.3 Liposomes

Liposomes can be produced by standard methods such as those reported by Kim et al. (1983, Biochim. Biophys. Acta 728, 339-348); Liu et al. (1992, Biochim. Biophys. Acta 1104, 95-101); Lee et al. (1992, Biochim. Biophys. Acta. 1103, 185-197), Brey et al. (U.S. Pat. Appl. Pub. 20020041861), Hass et al. (U.S. Pat. Appl. Pub. 20050232984), Kisak et al. (U.S. Pat. Appl. Pub. 20050260260) and Smyth-Templeton et al. (U.S. Pat. Appl. Pub. 20060204566). Additionally, reference may be made to Copeland et al. (2005, Immunol. Cell Biol. 83: 95-105) who review lipid based particulate formulations for the delivery of antigen, and to Bramwell et al. (2005, Crit Rev Ther Drug Carrier Syst. 22(2):151-214; 2006, J Pharm Pharmacol. 58(6):717-728) who review particulate delivery systems for vaccines, including methods for the preparation of protein-loaded liposomes. Many liposome formulations using a variety of different lipid components have been used in various in vitro cell culture and animal experiments. Parameters have been identified that determine liposomal properties and are reported in the literature, for example, by Lee et al. (1992, Biochim. Biophys. Acta. 1103, 185-197); Liu et al. (1992, Biochim. Biophys. Acta. 1104, 95-101); and Wang et al. (1989, Biochem. 28, 9508-951).

Briefly, the lipids of choice (and any organic-soluble bioactive), dissolved in an organic solvent, are mixed and dried onto the bottom of a glass tube under vacuum. The lipid film is rehydrated using an aqueous buffered solution containing any water-soluble bioactives to be encapsulated by gentle swirling. The hydrated lipid vesicles can then be further processed by extrusion, submitted to a series of freeze-thawing cycles or dehydrated and then rehydrated to promote encapsulation of bioactives. Liposomes can then be washed by centrifugation or loaded onto a size-exclusion column to remove unentrapped bioactive from the liposome formulation and stored at 4° C. The basic method for liposome preparation is described in more detail in Thierry et al. (1992, Nuc. Acids Res. 20:5691-5698).

A particle carrying a payload of bioactive agent(s) can be made using the procedure as described in: Pautot et al. (2003, Proc. Natl. Acad. Sci. USA 100(19):10718-21). Using the Pautot et al. technique, streptavidin-coated lipids (DPPC, DSPC, and similar lipids) can be used to manufacture liposomes. The drug encapsulation technique described by Needham et al. (2001, Advanced Drug Delivery Reviews 53(3): 285-305) can be used to load these vesicles with one or more active agents.

The liposomes can be prepared by exposing chloroformic solution of various lipid mixtures to high vacuum and subsequently hydrating the resulting lipid films (DSPC/CHOL) with pH 4 buffers, and extruding them through polycarbonated filters, after a freezing and thawing procedure. It is possible to use DPPC supplemented with DSPC or cholesterol to increase encapsulation efficiency or increase stability, etc. A transmembrane pH gradient is created by adjusting the pH of the extravesicular medium to 7.5 by addition of an alkalinization agent. A bioactive agent (e.g., a HDM and optionally an antigen to which a tolerogenic response is desired) can be subsequently entrapped by addition of a solution of the bioactive agent in small aliquots to the vesicle solution, at an elevated temperature, to allow accumulation of the bioactive agent inside the liposomes.

Other lipid-based particles suitable for the delivery of the bioactive agents of the present invention such as niosomes are described by Copeland et al. (2005, Immunol. Cell Biol. 83: 95-105).

6.1.4 Ballistic Particles

The D-amino acid peptides of the present invention may be attached to (e.g., by coating or conjugation) or otherwise associated with particles suitable for use in needleless or “ballistic” (biolistic) delivery. Illustrative particles for ballistic delivery are described, for example, in: International Publications WO 02/101412; WO 02/100380; WO 02/43774; WO 02/19989; WO 01/93829; WO 01/83528; WO 00/63385; WO 00/26385; WO 00/19982; WO 99/01168; WO 98/10750; and WO 97/48485. It shall be understood, however, that such particles are not limited to their use with a ballistic delivery device and can otherwise be administered by any alternative technique (e.g., injection or microneedle delivery) through which particles are deliverable to immune cells.

The bioactive agents can be coated or chemically coupled to carrier particles (e.g., core carriers) using a variety of techniques known in the art. Carrier particles are selected from materials which have a suitable density in the range of particle sizes typically used for intracellular delivery. The optimum carrier particle size will, of course, depend on the diameter of the target cells. Illustrative particles have a size ranging from about 0.01 to about 250 μm, from about 10 to about 150 μm, and from about 20 to about 60 μm; and a particle density ranging from about 0.1 to about 25 g/cm3, and a bulk density of about 0.5 to about 3.0 g/cm3, or greater. Non-limiting particles of this type include metal particles such as, tungsten, gold, platinum and iridium carrier particles. Tungsten particles are readily available in average sizes of 0.5 to 2.0 μm in diameter. Gold particles or microcrystalline gold (e.g., gold powder A1570, available from Engelhard Corp., East Newark, N.J.) may also be used. Gold particles provide uniformity in size (available from Alpha Chemicals in particle sizes of 1-3 μm, or available from Degussa, South Plainfield, N.J. in a range of particle sizes including 0.95 μm) and low toxicity. Microcrystalline gold provides a diverse particle size distribution, typically in the range of 0.1-5 μm. The irregular surface area of microcrystalline gold provides for highly efficient coating with the active agents of the present invention.

Many methods are known and have been described for adsorbing, coupling or otherwise attaching bioactive molecules (e.g., hydrophilic molecules such as proteins and nucleic acids) onto particles such as gold or tungsten particles. In illustrative examples, such methods combine a predetermined amount of gold or tungsten with the bioactive molecules, CaCl₂) and spermidine. In other examples, ethanol is used to precipitate the bioactive molecules onto gold or tungsten particles (see, for example, Jumar et al., 2004, Phys Med. Biol. 49:3603-3612). The resulting solution is suitably vortexed continually during the coating procedure to ensure uniformity of the reaction mixture. After attachment of the bioactive molecules, the particles can be transferred for example to suitable membranes and allowed to dry prior to use, coated onto surfaces of a sample module or cassette, or loaded into a delivery cassette for use in particular particle-mediated delivery instruments.

The formulated compositions may suitably be prepared as particles using standard techniques, such as by simple evaporation (air drying), vacuum drying, spray drying, freeze drying (lyophilization), spray-freeze drying, spray coating, precipitation, supercritical fluid particle formation, and the like. If desired, the resultant particles can be dandified using the techniques described in International Publication WO 97/48485.

6.1.5 Surfactants

Surfactants which can be incorporated into particles include phosphoglycerides. Exemplary phosphoglycerides include phosphatidylcholines, such as the naturally occurring surfactant, L-α-phosphatidylcholine dipalmitoyl (“DPPC”). The surfactants advantageously improve surface properties by, for example, reducing particle-particle interactions, and can render the surface of the particles less adhesive. The use of surfactants endogenous to the lung may avoid the need for the use of non-physiologic surfactants.

Providing a surfactant on the surfaces of the particles can reduce the tendency of the particles to agglomerate due to interactions such as electrostatic interactions, Van der Waals forces, and capillary action. The presence of the surfactant on the particle surface can provide increased surface rugosity (roughness), thereby improving aerosolization by reducing the surface area available for intimate particle-particle interaction.

Surfactants known in the art can be used including any naturally occurring surfactant. Other exemplary surfactants include diphosphatidyl glycerol (DPPG); hexadecanol; fatty alcohols such as polyethylene glycol (PEG); polyoxyethylene-9-lauryl ether; a surface active fatty acid, such as palmitic acid or oleic acid; sorbitan trioleate (Span 85); glycocholate; surfactin; a poloxamer; a sorbitan fatty acid ester such as sorbitan trioleate; tyloxapol and a phospholipid.

In order that the invention may be readily understood and put into practical effect, particular preferred embodiments will now be described by way of the following non-limiting examples.

### EXAMPLES

### Example 1

### Production of the Synthetic Peptide Combinatorial Library

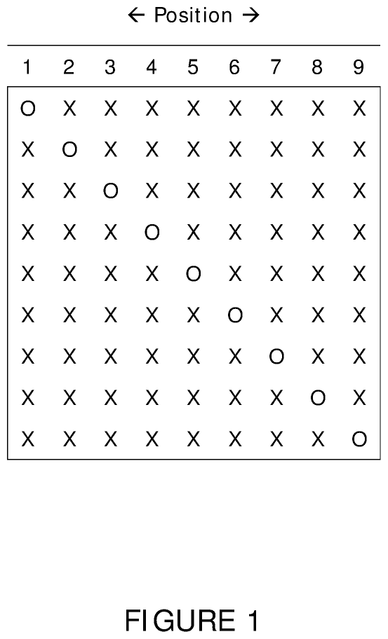

In order to provide a system that allows a robust and reproducible testing of experimental synthetic T-cell agonists in human cells in vitro, a combinatorial peptide library using synthetic D-amino acids (“D-CPL”) was generated as a novel platform for identifying synthetic ligands for use as T-cell vaccines. First, a nine-residue (nonamer) D-CPL was manufactured according to prior art methods using the simultaneous multiple peptide synthesis method (see, Pinilla et al., Biotechniques, 13: 901-905, 1992). The library consisted of 180 mixtures in the OX₈ format, where O represents one each of the 20 natural D-amino acids (i.e., a, c, d, e, i, f, g, h, k, l, m, n, p, q, r, s, t, v, w and y) at a defined position and X represents any of the 20 D-amino acids (with the exception of cysteine), in each of the remaining positions (see, FIG. 1).

The first mixture had D-alanine (a) in position 1 (a₁X₈), while mixture number 180 had D-tyrosine (γ) in position 0 (x₈y₉). Each OX mixture therefore consisted of 1.7×10¹⁰ (19⁸) different nonamer peptides in approximately equimolar concentration, and the total X₉ library consisted of 3.4×10¹¹ (9×20×19⁸) different peptides. Assuming an average molecular weight of 1080 Da for each nonamer and a concentration of 100 μg/mL (93 μM) for nonapeptides in a mixture, the average concentration of individual nonapeptides in a mixture was about 5.5×10⁻¹⁵.

### Materials and Methods

D-amino acid nonamer CPLs in positional scanning format (Pepsan Presto; and as previously described for L-peptide synthesis in Wooldridge, et al., 2010) were made to high purity using solid phase peptide synthesis and high-performance liquid chromatography (Pepscan Presto and GL Biochem).

### Example 2

### Identification of D-Amino Acid Agonists

The influenza A model was used to obtain proof of concept data, as this model allows human T-cell memory to be clearly examined in vitro with the benefit of a pathogen that can infect both humans and humanized mice. The blueprint for synthetic agonist design was the immunodominant HLA-A*0201-restricted GILGFVFTL₅₈₋₆₆ (“GIL”) peptide (HLA-A2-GIL) from the Matrix M1 protein.

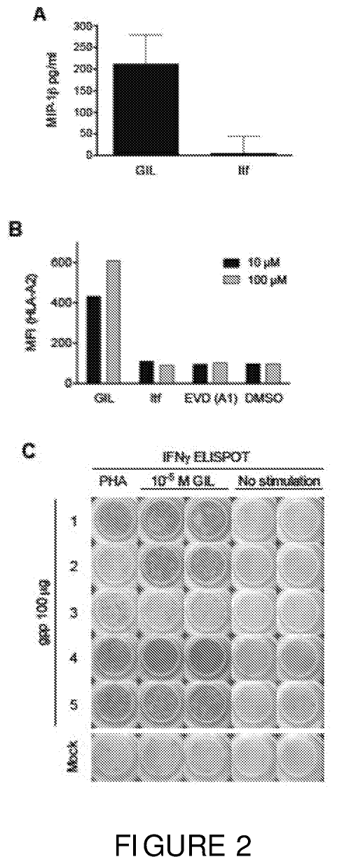

As a first experiment, the ability of a retro-inversion of the GIL epitope (i.e., ltfvfglig; (“ltf”), with lower case letters used to denote D-amino acids) to activate an archetypal TRBV19-positive (see, Moss et al. 1991) HLA-A2-GIL specific CD8⁺ T-cell clone ALF3 was examined. This experiment was performed as D-amino acid retro-inversions have been known to occasionally cross-react with their native L-amino acid counterparts (reviewed in Peniter, et al., 2013). Direct comparisons of HLA-A2-GIL versus HLA-A2-ltf activation on the ALF3 clone revealed that retro-inversion was not immunogenic (FIG. 2A), an outcome likely influenced by the weak binding of ltf within the binding cleft of HLA-A2 (FIG. 26).

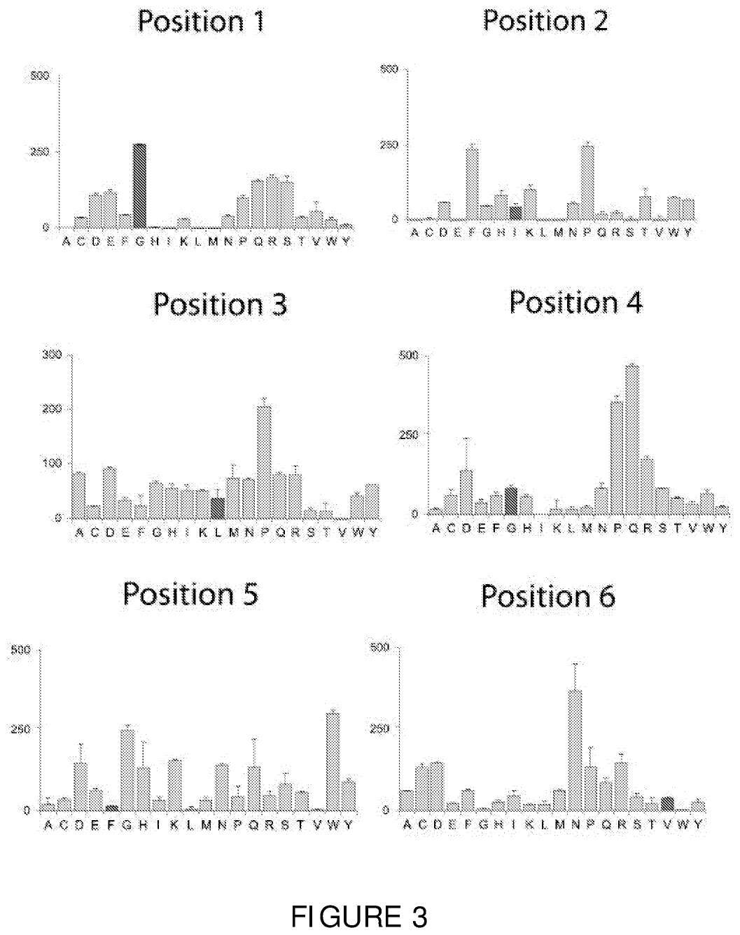

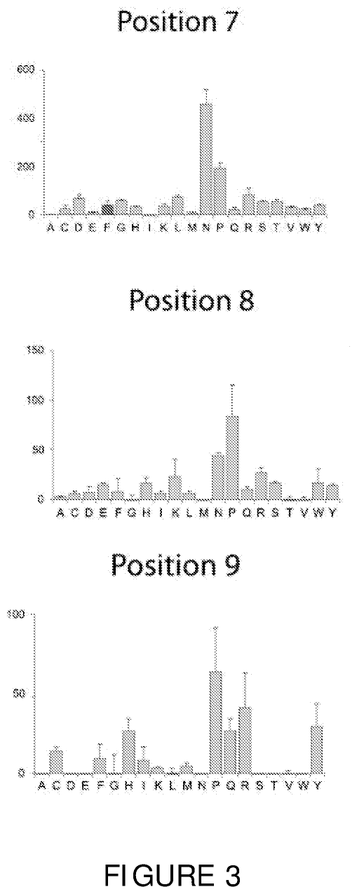

CPL scanning allows a systematic search for non-natural D-amino acid agonists that are capable of triggering HLA-A2-GIL-specific CD8⁺ T-cells. Essentially, this platform for quantitative and unbiased determination of TCR amino acid preference(s) across the full backbone of MHC-bound peptide and is a powerful tool to identify and/or augment T-cell agonists. Given the success in using CPL platforms to generate L-amino acid-based pMHC-I agonists in the pathogen, cancer, and autoimmune settings (Wooldridge et al., 2012; Ekeruche-Makinde et al., 2013; Lau, et al., 2014; and Ekeruche-Makinde et al., 2012), it was hypothesized that a non-natural D-amino acid based CPL could elicit similar signals and outcomes. To test this, a novel D-amino acid nonamer CPL was synthesized (see, Example 1) and used in positional scanning format to stimulate the ALF3 T-cells using a fully quantitative MIP-1β ELISA readout (FIG. 3). ALF3 was selected to represent a common bias toward TRBV19 gene usage within GIL-specific memory (Moss et al., 1991; Lehner et al., 1995; and Neller et al., 2015). Of note, the D-amino acid CPL was length-matched to the GIL peptide, given previous data showing that MHC-I-restricted TCRs are pre-programmed to engage bound ligands spanning a defined number of residues (see, Ekeruche-Makinde et al., 2013). In contrast to the traditional L-amino acid CPL scan, that revealed a limited recognition profile across most of the 180 peptide mixtures, the D-CPL scan indicated that every residue along the peptide backbone could potentially be substituted with several different D-amino acids. Indeed, the scan suggests ALF3 T-cells may recognize many more D-amino acid agonists compared with L-amino acid agonists, further highlighting the vast cross-reactive potential of αβ TCR surveillance against non-classical antigens.

### Materials and Methods

### Human T Cell Clones and Target Cells

The CD8⁺ T cell clones ALF3 and GD were maintained in RPMI medium supplemented with 100 IU/mL penicillin, 100 μg/mL streptomycin, 2 mM L-glutamine and 10% heat-inactivated fetal calf serum (R10), together with 25 ng/mL IL-15 (PeproTech), 200 IU/mL IL-2 (Proleukin) and 2.5% Cellkines (Helvetica Healthcare). C1R-HLA-A*0201 (C1R-A2) cells were generated as described previously and maintained in R10. C1R-A2 cells were also lentivirally transduced to express the M1 protein from influenza A virus H1N1 strain A/Puerto Rico/8/34 (PR8). The T-lymphoblastoid hybrid cell line 0.174×CEM.T2 (T2) was maintained in R10.

### In Vitro Expansion of Human T Cells

PBMCs were isolated by standard density gradient centrifugation from locally sourced venous blood samples or buffy packs obtained from the Welsh Blood Service, UK. All donors provided informed consent in accordance with institutional guidelines. Cryopreserved PBMCs were stimulated with peptide at the indicated concentrations in R10. Progressively greater concentrations of IL-2 were added from day 2 to a maximum of 20 IU/mL by day 14. The cultures were then analyzed and sorted by flow cytometry.

### CPL Scans

For CPL screening, CD8⁺ T-cell clones were rested overnight in R2 (as for R10 but with 2% FBS). Target cells (6×10⁴ per well) were incubated in 96-well U-bottom plates with library mixtures (at 100 μM) in duplicate for 2 hours at 37° C. Subsequently, 3×10⁴ clonal CD8⁺ T-cells were added and the assay incubated overnight at 37° C. The supernatant was then harvested and assayed for MIP-1β by ELISA according to the manufacturer's instructions (R&D Systems).

Conditions and analysis for the titration of individual peptides were performed as for the CPL using the peptide concentrations specified. Additionally, functional sensitivity for individual peptides is expressed by the pEC₅₀ of that peptide with respect to the TCR. This is defined as minus 1 times the base-10 logarithm of the 50% efficacy concentration such that increases in functional sensitivity translate into increases in the pEC₅₀ value. pEC₅₀ values were estimated as previously described (see, Wooldridge, L., Ekeruche-Makinde, J., van den Berg, H. A., Skowera, A., Miles, J. J., et al., (2012) A single autoimmune T-cell receptor recognizes more than a million different peptides, J. Biol. Chem., 287: 1168-1177).

### Example 3

### Selection of Recognized Residues to Prepare T-Cell Agonist

Informed by the quantitative data described in Example 2, we designed and synthesized eight D-amino acid peptide agonists for competitive testing in functional experiments. The peptides generated are listed in the following table:

| D-amino acid | SEQ ID |
|---|---|
| sequence | # |
| gppqwnnpp | 3 |
| rfpqwnnpp | 4 |
| rfpqgnnpp | 5 |
| rfppwnnpp | 6 |
| gpppwnnpp | 7 |
| gpppgnnpp | 8 |
| rfppgnnpp | 9 |
| gppqgnnpp | 10 |

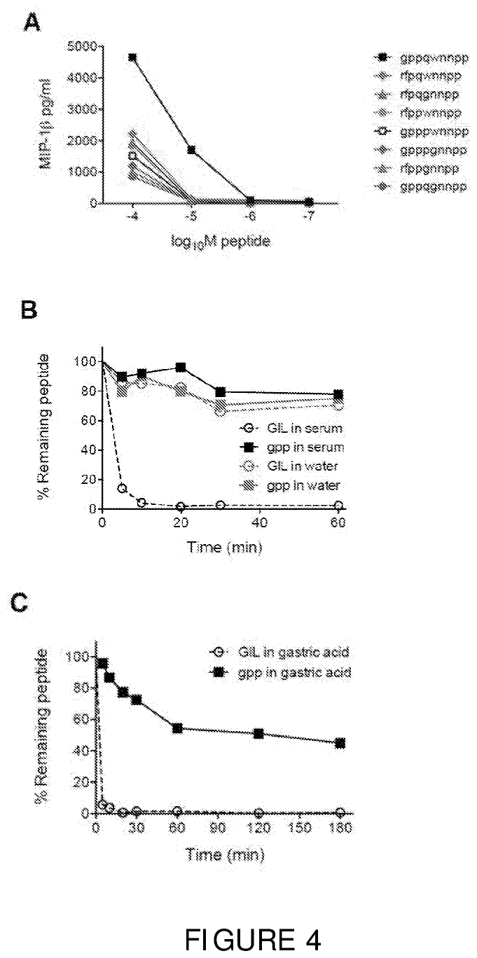

Dose-response titrations using MIP-1β production as a readout showed that gppqwnnpp (gpp), SEQ ID NO:3, was the most potent activator of ALF3 (FIG. 4A). The gpp sequence incorporated the dominant residue in terms of signal strength at each index position. It is also notable that gpp bears no resemblance to GIL in terms of primary sequence, barring the N-terminal glycine residue, for which there is no D-amino acid counterpart. This observation demonstrates the power of combinatorial screening as a means to identify unrelated agonists.

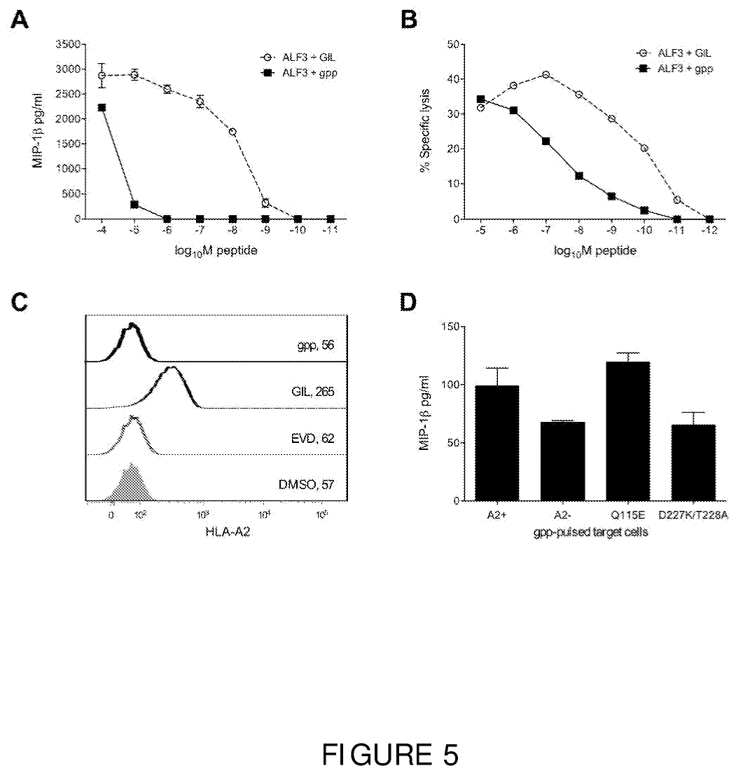

Next, we compared the ability of each agonist to trigger effector outputs in peptide titration experiments with ALF3 T-cells. Strong activation was observed in response to GIL, both in terms of MIP-1β production (FIG. 5A) and target cell killing (FIG. 5B). Similarly, gpp elicited potent responses, but higher concentrations of the agonist were required for each function. We hypothesized that the lower functional sensitivity of gpp may reflect a lack of traditional HLA-A2 anchor residues in the D-amino acid sequence, thereby destabilizing the binary pMHC-I complex. Using a T2 peptide binding assay (Miles et al., 2011), we observed weak upregulation of HLA-A2 in the presence of gpp (FIG. 5C), with no improvement following the addition of exogenous β2-microglobulin (data not shown) (Nijman et al., 1993). Given the limited dynamic range of this assay (Miles et al., 2011), we sought to confirm epitope presentation in a transgenic cell system (FIG. 5D) (Purbhoo et al., 2001; Wooldridge et al., 2005; and Wooldridge et al., 2007). We found that gpp-pulsed HLA-A2⁺ CIR (C1R-A2) targets, but not gpp-pulsed HLA-A2⁻ CIR targets, could activate ALF3. In addition, C1R-A2 targets with enhanced CD8 binding due to a Q115E mutation in the α2 domain of HLA-A2 were effective presenting cells for gpp, whereas C1R-A2 targets with abrogated CD8 binding due to a compound D227K/T228A mutation in the α3 domain of HLA-A2 did not enable gpp to activate ALF3. These observations show that gpp is restricted by HLA-A2 and elicits functional outputs that are dependent on CD8.

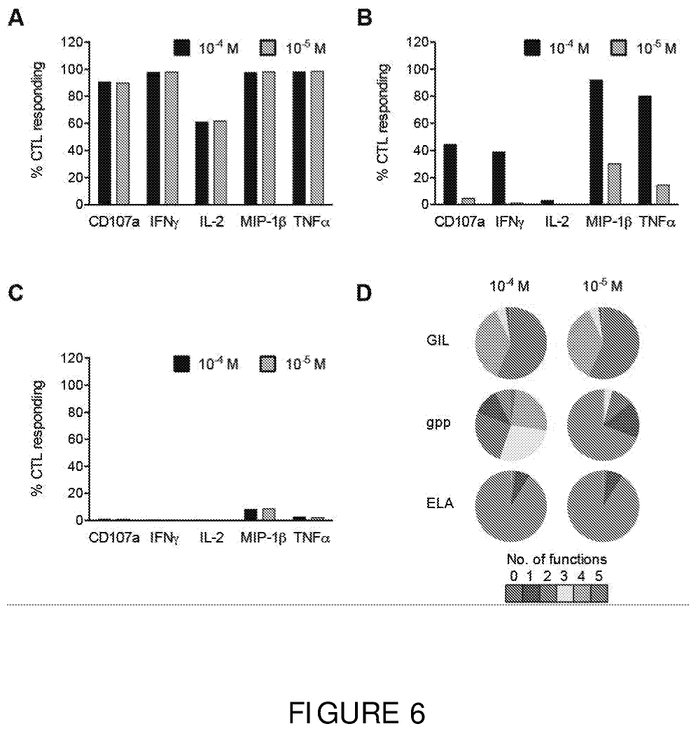

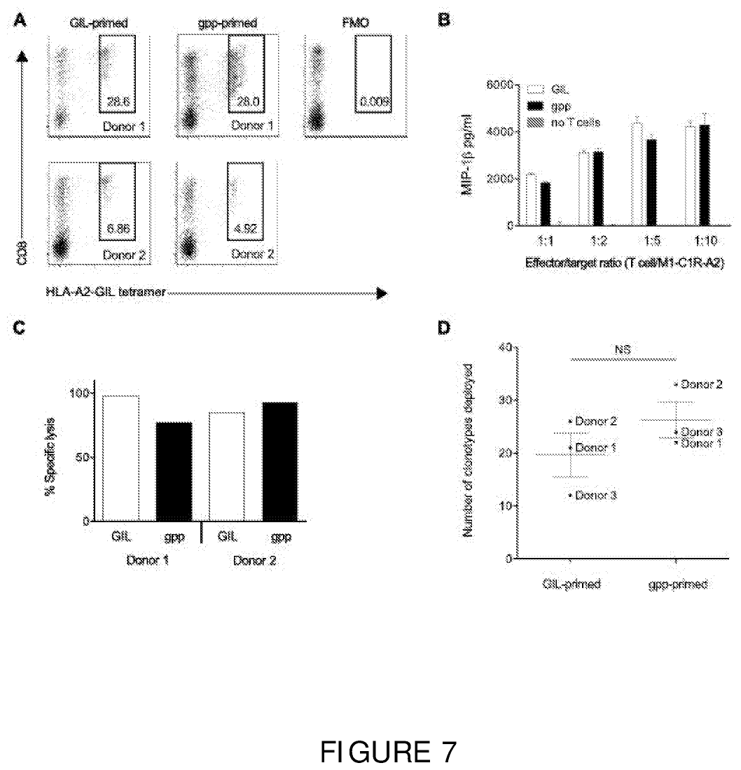

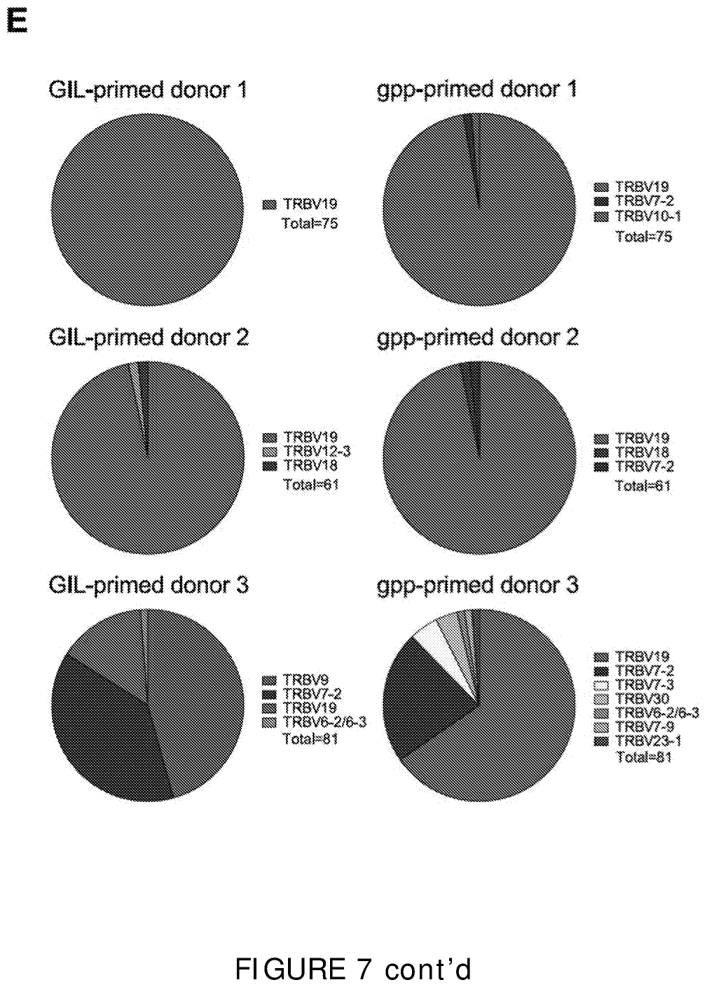

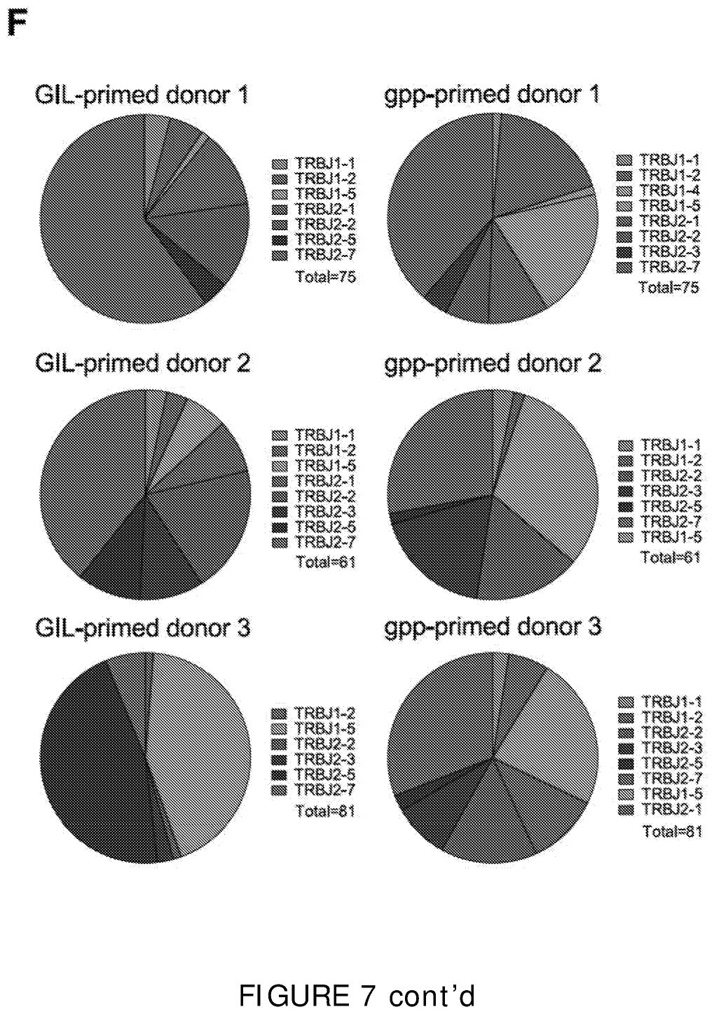

In further experiments, we used intracellular cytokine staining to examine the agonist-induced functional profile of another TRBV19⁺CD8⁺ T-cell clone (GD). Five different effector outputs (CD107a, IFNγ, IL-2, MIP-1β and TNFα) were measured simultaneously by flow cytometry in response to two different concentrations of gpp and GIL (FIG. 6A-D), at 10⁻⁴ M, gpp elicited multiple functions, with >80% of clonal GD cells expressing both MIP-1β and TNFα. In line with the cytokine release and cytotoxicity data, however, a weaker profile was observed at 10⁻⁵ M. This loss of sensitivity likely relates to the weak affinity of gpp for HLA-A2. In contrast, the native GIL peptide elicited highly polyfunctional responses at 10⁻⁴ and 10⁻⁵ M.

To extend these observations, we investigated the ability of gpp to amplify GIL-specific human memory T-cells in vitro. Peripheral blood mononuclear cells (PBMCs) from healthy HLA-A2⁺ individuals were stimulated with either gpp or GIL for 10 days, and specific T-cell expansions were quantified by flow cytometry after staining with a fluorochrome-labeled tetrameric HLA-A2-GIL complex (FIG. 5A). Remarkably, we found that both gpp and GIL primed comparable populations of tetramer-binding CD8⁺ T-cells. In addition, we observed equivalent functional reactivity against C1R-A2 target cells expressing the full-length influenza A virus M1 protein (FIGS. 5B&C). These data show that gpp can expand GIL-specific memory CD8⁺ T-cells capable of recognizing the endogenously processed L-amino acid target antigen in the context of HLA-A2.

### Materials and Methods

pMHC-I Tetramer Staining

Soluble biotinylated pMHC-I monomers were produced as described previously (Lissina et al., 2009). Tetrameric pMHC-I reagents (tetramers) were constructed by the addition of APC-conjugated streptavidin (Life Technologies) at a pMHC-I:streptavidin molar ratio of 4:1. CD8⁺ T-cell clones or bulk cultures (5×10⁴) were incubated with APC-labeled tetramer (25 μg/mL) for 15 min at 37° C. after staining with LIVE/DEAD Fixable Aqua (Life Technologies). Data were acquired using a FACSCantoII flow cytometer (BD Biosciences) and analyzed with FlowJo software (Tree Star Inc.).

Intracellular Cytokine Staining

T-cells were rested overnight at 1×10⁶ per mL in R2 and added to peptide-pulsed targets at an effector:target ratio of 1:2 in the presence of 5 μg/mL brefeldin A (Sigma-Aldrich), 0.35 μL/mL monensin (BD Biosciences) and 5 μL/mL αCD107a-FITC (BD Biosciences). After 5 h at 37° C., the cells were washed and stained with LIVE/DEAD Fixable Aqua (Life Technologies) followed by αCD3-PacificBlue, αCD8-APCH7 and αCD19-BV521 (BioLegend). The cells were then fixed/permeabilized using a Cytofix/Cytoperm Kit (BD Biosciences) and stained intracellularly with αIFNγ-PECγ7, αTNFα-PerCPCy5.5 and αIL-2-APC (BioLegend), and αMIP-1β-PE (BD Biosciences). Data were acquired using a FACSCantoII flow cytometer (BD Biosciences) and analyzed with FlowJo software (Tree Star Inc.). Cell population gates were set using fluorescence minus one staining controls as described previously (Firat et al., 1999).

Cytotoxicity Assays

⁵¹Cr release assays were performed as described previously (Tungatt et al., 1992) using HLA-A2⁺ T2 or M1-C1R-HLA-A2 cells as targets. Flow cytometric assays were performed using carboxyfluorescein diacetate succinimidyl ester (CFSE)-labeled targets. C1R-A2 cells (3×10⁴ per well) were either pulsed with 10⁻⁶ M GIL peptide for 1 h at 30° C. and labeled with 0.2 μL CFSE for 10 min to generate CFSE^{hi} targets or left unpulsed and labeled with 0.02 μl CFSE for 10 min to generate CFSE^{lo} controls. Targets and controls were incubated in the presence or absence of T-cells at an effector:target ratio of 5:1 in 96-well U-bottom plates. The assays were incubated overnight at 37° C. After harvesting, the cells were stained with LIVE/DEAD Fixable Aqua (Life Technologies) and αCD8-APC (BD Biosciences) before acquisition on a FACSCantoII flow cytometer (BD Biosciences). Data were analyzed with FlowJo software (Tree Star Inc.). Peptide-specific lysis was determined as the percent loss of CFSE^{hi} targets relative to CFSE^{lo} controls in the presence of T-cells.

Clonotype Analysis

Viable tetramer-positive CD3⁺CD8⁺ cells were sorted at >98% purity using a custom-modified FACSAriaII flow cytometer (BD Biosciences). Molecular analysis of expressed TRB gene rearrangements was conducted using a template-switch anchored RT-PCR with Sanger sequencing technology as described previously (Quigley et al., 2011).

### Example 4

### Protease and Acid Resistance of Native Versus Synthetic Agonists

To elicit immune responses in vivo, antigenic structures must navigate host barriers associated with the route of entry, such as serum complement/proteases, gastric acid and digestive enzymes. It is pertinent to note in this regard that strings of D-amino acids are thought to be sterically incompatible with protease-induced hydrolysis (Zhao and Lu, 2014). We compared the stability of gpp and GIL in human serum and simulated gastric acid as potential indicators of long-term biostability and immunogenicity. GIL was rapidly degraded in human serum, reaching almost undetectable levels within 10 minutes (FIG. 4B). In contrast, gpp remained largely intact after one hour in human serum. Similar disparities were observed in simulated gastric acid (FIG. 4C). These observations indicate that gpp is likely to be highly stable in vivo, potentially enabling more effective immune priming relative to GIL.

### Materials and Methods

Human serum from AB plasma (Sigma-Aldrich) was centrifuged for 10 min at 20,000 RCF to remove the lipid component. Serum supernatant was diluted to 25% in MilliQ water and incubated for 15 min at 37° C. Triplicate samples of native and synthetic peptides (>93% pure; GL Biochem) were assayed simultaneously at a final concentration of 50 μg/mL after 1:20 dilution with 25% serum. Control reactions were set up as single tests with peptides diluted to the same concentration in MilliQ water. Assays were run for the indicated times at 37° C. Samples of each peptide solution (100 μL) were then removed and mixed with an equal volume of 15% aqueous trichloroacetic acid to precipitate serum proteins. Reactions were incubated for 40 min at 4° C. and centrifuged for 5 min at 14,000 RCF. Supernatant was then stored at −20° C. before analysis by liquid chromatography-mass spectrometry (LCMS). Three distinct ion fragments were monitored for each peptide. Stability was calculated as the area percentage of each serum-treated ion peak relative to the same ion peak at 0 min. Simulated gastric acid was prepared by dissolving 20 mg of NaCl and 16 mg of porcine pepsin (Sigma-Aldrich) in 70 μL of concentrated HCl and diluting the solution to 10 mL with water (final pH 1.2). Mixtures were incubated for 15 min at 37° C. Triplicate samples of native and synthetic peptides were assayed as described above with dilution in simulated gastric acid. Control reactions were set up as single tests with peptides diluted to the same concentration in simulated gastric acid without pepsin. Assays were run for the indicated times at 37° C. Samples of each peptide solution (100 μL) were then removed and stored at −20° C. before analysis by LCMS. Data in percentages were square-root transformed for all assays. Statistical analyses were conducted using unpaired t-tests (one per time point) corrected for multiple comparisons using the Holm-Sidak method (Alpha 0.05) with Prism 6 (GraphPad Software Inc.).

### Example 5

### Structural Conformation of Native Versus Synthetic Agonists

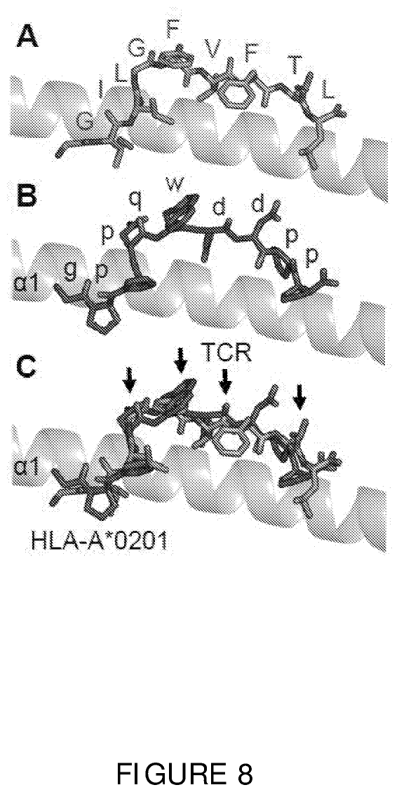

To determine the molecular basis of agonist cross-recognition in this setting, we attempted to solve the binary structure of the HLA-A2-gpp complex. Although refolded protein yields were very low, presumably reflecting the weak affinity of gpp for HLA-A2, we were able to generate small crystals. However, these crystals were not capable of diffracting to atomic resolution. We therefore modelled the HLA-A2-gpp structure in silico (FIGS. 8A&B), using the JM22-HLA-A2-GIL ternary complex as a guide (Stewart-Jones et al., 2003). The model indicated that gpp could be presented by HLA-A2 in a overall conformation similar to that of GIL. In particular, the D-amino acid residues Glu4, Trp5, Asp6 and Pro8 were solvent exposed, mimicking in three dimensions the main TCR contact residues identified in the JM22-HLA-A2-GIL complex (FIG. 8C) (Stewart-Jones et al., 2003). Thus, despite a lack of sequence homology between gpp and GIL, both antigens may look similar in terms of shape complementarity.

### Materials and Methods

The structure of gpp complexed with HLA-A2 was modelled in WinCoot using the JM22 TCR-HLA-A2-GIL ternary structure as a reference (see, Stewart Jones et al., 2003). The model was regularized using REFMAC5.

### Example 6

### The Synthetic Agonist Effective Primes T-Cell Response that can Protect from Lethal Influenza Challenge

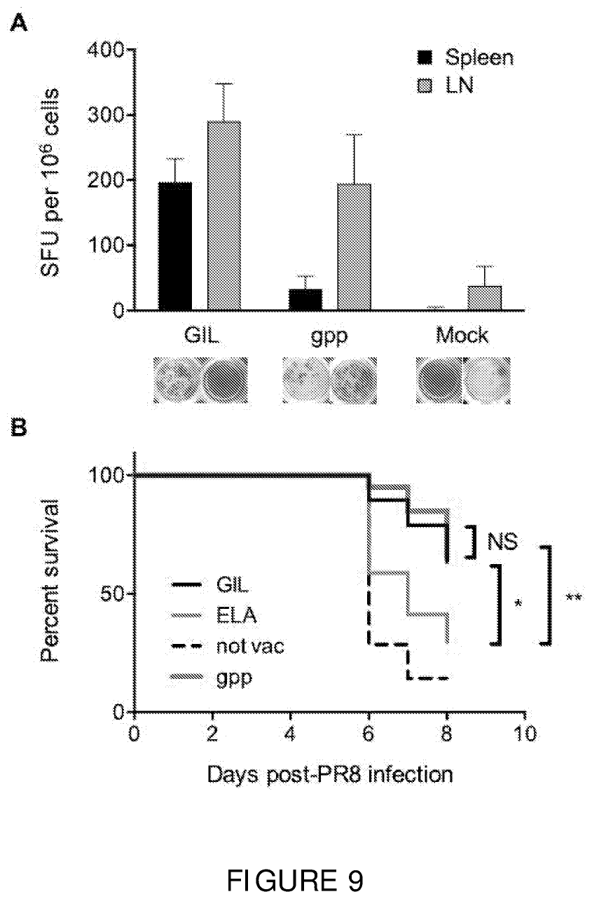

To assess the biological relevance of these observations, we tested the ability of gpp to prime effective de novo responses from the naïve T-cell pool. We used transgenic HLA-A2 HHD mice for this purpose, based on earlier work in similar transgenic murine systems (Plotnicky et al., 2003). Mice were injected on day 0 and day 14 with GIL, gpp or an irrelevant HLA-A2-restricted L-amino acid peptide (ELAGIGILTV) in Incomplete Freund's Adjuvant. Preliminary dosing experiments showed that gpp was safe and non-toxic (data not shown). One week after the second injection, cells were harvested from the spleen and peripheral lymph nodes (PLNs). Using direct ex vivo IFNγ ELISPOT analysis, we found that gpp induced a GIL-specific response in vivo, detectable most prominently in the PLNs (FIG. 9A). No such response was observed with the ltf retro-inversion of GIL (data not shown).

Next, mice were immunized with the same regimen and subsequently infected with influenza virus strain H1N1 A/PR/8/34 (PR8) intranasally on day 21. On day 6 post-PR8 infection, mice vaccinated with the control ELAGIGILTV peptide began to succumb rapidly (FIG. 9B). In contrast, mice vaccinated with either GIL or gpp fared significantly better, with survival rates >60% at day 8. It is also notable that a trend was observed towards better outcomes in the gpp versus GIL groups.

To extend these findings, we assessed the immunogenic effects of orally administered gpp, which is stable in simulated gastric acid (FIG. 4). Mice received three doses of non-adjuvanted gpp (300 μg total) in sodium bicarbonate at weekly intervals via oral gavage. One week after the final dose, cells were harvested from the mesenteric lymph nodes and tested for GIL reactivity using IFNγ ELISPOT assays (FIG. 2C). Substantial GIL-specific responses were detected in gpp-vaccinated mice but not in mock-vaccinated controls. Collectively, these experiments demonstrate that gpp can prime protective immune responses in a humanized mouse model of influenza virus infection.

### Materials and Methods

Mice

All mouse experiments were conducted in accordance with the UK Animals (Scientific Procedures) Act 1986 under Project Licences 30/2355, 30/2635 and 30/3188. HHD mice were provided as a kind gift by Immunocore Ltd. (Didcot, UK) or purchased from the Weatherall Institute of Molecular Medicine at the University of Oxford (Oxford, UK). These mice express a hybrid HLA-A2 transgene comprising the human α1/α2 domains and β2-microglobulin fused with a murine α3 domain (H-2Db) [57,58]. Mice were housed throughout under specific pathogen-free conditions.

### Immunization, Organ Harvest and Influenza Infection

HHD mice were primed in the ventral inguinal area by injection with 200 μL of a PBS preparation containing 100 μg of peptide (GIL, gpp or ELA) and 100 μL of Incomplete Freund's Adjuvant (Sigma-Aldrich). The same preparation was used to boost on the contralateral side 14 days later. Care was taken to ensure the formation of a raised area at the injection site, indicating that the vaccine was delivered subcutaneously rather than intraperitoneally. For experiments involving organ harvest, mice were euthanized 7 days after the last immunization and the peripheral lymph nodes (inguinal, axial, brachial and submandibular) were prepared as single cell suspensions. For challenge experiments, mice were infected intranasally with influenza A virus strain PR8 obtained from the National Institute for Medical Research (London, UK). On the basis of dose optimization experiments, male mice received 100 plaque forming units (PFU) and female mice received 50 PFU of PR8 in 50 μl of sterile PBS under light anesthesia. Body weight was recorded daily after infection. Mice were classified as non-survivors and euthanized if their body weight fell by >20%. All other mice were euthanized 8 days after infection.

Mouse Interferon-γ ELISPOT

IFNγ-producing cells were quantified using a Mouse IFNγ ELISPOT Kit (MabTech). Briefly, ELISPOT multiscreen filter plates (Millipore) were coated with capture antibody for 4 h at 37° C., then washed with PBS and blocked with R10 for 1 h at room temperature. Cells were added at 2×10⁵ per well in the presence of peptide at a final concentration of 10-5 M. Medium only was used as a negative control and PHA (1 μg/mL) was used as a positive control. Assays were incubated overnight at 37° C., and the plates were developed as per the manufacturer's instructions (MabTech). Spot-forming-units (SFU) were counted using an AID ELISPOT Reader v5 (AID Diagnostika GmbH).

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

### BIBLIOGRAPHY

Birnbaum M E, Mendoza J L, Sethi D K, Dong S, Glanville J, et al. (2014) Deconstructing the peptide-MHC specificity of T-cell recognition. Cell 157: 1073-1087.
Bridgeman J S, Sewell A K, Miles J J, Price D A, Cole D K (2012) Structural and biophysical determinants of alphabeta T-cell antigen recognition. Immunology 135: 9-18.
Chen X, Fernando G J, Crichton M L, Flaim C, Yukiko S R, et al. (2011) Improving the reach of vaccines to low-resource regions, with a needle-free vaccine delivery device and long-term thermostabilization. J. Contro.l Release 152: 349-355.
Cui Z, Sun L, Zhang B. (2004) A peptidyl transferase ribozyme capable of combinatorial peptide synthesis. Bioorg Med Chem. 12(5): 927-33.
Ekeruche-Makinde J, Clement M, Cole D K, Edwards E S, Ladell K, et al. (2012) T-cell receptor-optimized peptide skewing of the T-cell repertoire can enhance antigen targeting. J. Biol. Chem. 287: 37269-37281.
Ekeruche-Makinde J, Miles J J, van den Berg H A, Skowera A, Cole D K, et al. (2013) Peptide length determines the outcome of TCR/peptide-MHCI engagement. Blood 121: 1112-1123.
Firat, H., Garcia-Pons, F., Tourdot, S., Pascolo, S., Scardino, A., et al., (1999) H-2 class I knockout HLA-A2.1-transgenic mice: a versatile animal model for preclinical evaluation of antitumour immunotherapeutic strategies. Eur. J. Immunol. 29: 2113-3121.
Fuchs S A, Berger R, Klomp L W, de Koning T J (2005) D-amino acids in the central nervous system in health and disease. Mol. Genet. Metab. 85: 168-180.
Lau L S, Fernandez-Ruiz D, Mollard V, Sturm A, Neller M A, et al. (2014) CD8+ T-cells from a novel T-cell receptor transgenic mouse induce liver-stage immunity that can be boosted by blood-stage infection in rodent malaria. PLoS Pathog. 10: e1004135.
Lehner P J, Wang E C, Moss P A, Williams S, Platt K, et al. (1995) Human HLA-A0201-restricted cytotoxic T lymphocyte recognition of influenza A is dominated by T-cells bearing the V beta 17 gene segment. J. Exp. Med. 181: 79-91.
Lissina, A, Ladell, K, Skowera, A, Clement, M, Edwards, E, et al., (2009) Protein kinase inhibitors substantially improve the physical detection of T-cells with peptide-MHC tetramers. J. Immunol. Methods., 340: 11-24.
Loeffler, F F, Foertsch, T C, Popov, R, Matte, D S, Schlageter M, Sedlmayr, M, Ridder B, Dang, F X, mens von Bojniĉić-Kninski, C, Weber, L K, Fischer A, Greifenstein, J, Bykovskaya, V, Buliev, I, Bischoff, F R, Hahn, L, Meier, Bräse, S, Powell, A K, Balaban, T S, Breitling, F, Nesterov-Mueller, A. (2016) High-flexibility combinatorial peptide synthesis with laser-based transfer of monomers in solid matrix material, Nat Comm, 7: 11844.
Miles J J, Douek D C, Price D A (2011) Bias in the alphabeta T-cell repertoire: implications for disease pathogenesis and vaccination. Immunol Cell Biol 89: 375-387.
Miles J J, McCluskey J, Rossjohn J, Gras S (2015) Understanding the complexity and malleability of T-cell recognition. Immunol. Cell Biol. 93: 433-441.
Miles K M, Miles J J, Madura F, Sewell A K, Cole D K (2011) Real time detection of peptide-MHC dissociation reveals that improvement of primary MHC-binding residues can have a minimal, or no, effect on stability. Mol. Immunol. 48: 728-732.
Moss P A, Moots R J, Rosenberg W M, Rowland-Jones S J, Bodmer H C, et al., (1991) Extensive conservation of alpha and beta chains of the human T-cell antigen receptor recognizing HLA-A2 and influenza A matrix peptide. Proc. Natl. Acad. Sci. USA, 88: 8987-8990.
Neller M A, Ladell K, McLaren J E, Matthews K K, Gostick E, et al. (2015) Naive CD8(+) T-cell precursors display structured TCR repertoires and composite antigen-driven selection dynamics. Immunol. Cell Biol. 93: 625-633.
Nijman H W, Houbiers J G, Vierboom M P, van der Burg S H, Drijfhout J W, et al. (1993) Identification of peptide sequences that potentially trigger HLA-A2.1-restricted cytotoxic T lymphocytes. Eur. J. Immunol. 23: 1215-1219.
Pentier J M, Sewell A K, Miles J J, (2013) Advances in T-cell epitope engineering. Front. Immunol., 4: 133.
Pinilla C, Martin R, Gran B, Appel J R, Boggiano C, et al. (1999) Exploring immunological specificity using synthetic peptide combinatorial libraries. Curr Opin Immunol 11: 193-202.
Plotnicky H, Cyblat-Chanal D, Aubry J P, Derouet F, Klinguer-Hamour C, et al. (2003) The immunodominant influenza matrix T-cell epitope recognized in human induces influenza protection in HLA-A2/K(b) transgenic mice. Virology 309: 320-329.
Pollegioni L, Piubelli L, Sacchi S, Pilone M S, Molla G (2007) Physiological functions of D-amino acid oxidases: from yeast to humans. Cell Mol Life Sci 64: 1373-1394.
Purbhoo M A, Boulter J M, Price D A, Vuidepot A L, Hourigan C S, et al. (2001) The human CD8 coreceptor effects cytotoxic T-cell activation and antigen sensitivity primarily by mediating complete phosphorylation of the T-cell receptor zeta chain. J. Biol. Chem. 276: 32786-32792.
Purcell A W, McCluskey J, Rossjohn J (2007) More than one reason to rethink the use of peptides in vaccine design. Nat Rev Drug Discov 6: 404-414.
Quigley, M. F., Mlmeida, J. R., Price, D. A., Douek, D. C., (2011) Unbiased molecular analysis of T cell receptor expression using template-switch anchored RT-PCR. Curr. Protoc. Immunol., Chpt. 10: Unit 10.33.
Sewell A K (2012) Why must T-cells be cross-reactive? Nat Rev Immunol 12: 669-677.
Stepanek O, Prabhakar A S, Osswald C, King C G, Bulek A, et al. (2014) Coreceptor scanning by the T-cell receptor provides a mechanism for T-cell tolerance. Cell 159: 333-345.
Stewart-Jones G B, McMichael A J, Bell J I, Stuart D I, Jones E Y (2003) A structural basis for immunodominant human T-cell receptor recognition. Nat. Immunol. 4: 657-663.
Strese S, Fryknas M, Larsson R, Gullbo J. (2013) Effects of hypoxia on human cancer cell line chemosensitivity. BMC Cancer 13:331.
Tan M P, Gerry A B, Brewer J E, Melchiori L, Bridgeman J S, et al. (2015) T-cell receptor binding affinity governs the functional profile of cancer-specific CD8+ T-cells. Clin Exp Immunol 180: 255-270.
Torres A M, Tsampazi C, Geraghty D P, Bansal P S, Alewood P F, et al. (2005) D-amino acid residue in a defensin-like peptide from platypus venom: effect on structure and chromatographic properties. Biochem J 391: 215-220.
Tungatt, K, Bianchi, V, Crowther, M D, Powell, W E. Schauenburg, A. J., et al, (2015) Antibody stabilization of peptide-MHC multimers reveals functional T-cells bearing extremely low affinity TCRs, J Immunol., 194: 463-474.
UNICEF (1996) UNICEF Vaccines bring 7 diseases under control. UNICEF Vaccines bring 7 diseases under control. http://www.unicef.org/pon96/hevaccin.htm: UNICEF
van den Berg H A, Ladell K, Miners K, Laugel B, Llewellyn-Lacey S, et al. (2013) Cellular-level versus receptor-level response threshold hierarchies in T-cell activation. Front. Immunol. 4: 250.
Wolfson L J, Gasse F, Lee-Martin S P, Lydon P, Magan A, et al. (2008) Estimating the costs of achieving the WHO-UNICEF Global Immunization Vision and Strategy, 2006-2015. Bull. World Health Organ. 86: 27-39.
Wooldridge L, van den Berg H A, Glick M, Gostick E, Laugel B, et al. (2005) Interaction between the CD8 coreceptor and major histocompatibility complex class I stabilizes T-cell receptor-antigen complexes at the cell surface. J. Biol. Chem. 280: 27491-27501.
Wooldridge L, Lissina A, Vernazza J, Gostick E, Laugel B, et al. (2007) Enhanced immunogenicity of CTL antigens through mutation of the CD8 binding MHC class I invariant region. Eur. J. Immunol. 37: 1323-1333.
Wooldridge L, Laugel B, Ekeruche J, Clement M, van den Berg, H A, et al., (2010) CD8 controls T-cell cross-reactivity, J. Immunol., 185: 4625-4632.
Wooldridge L, Ekeruche-Makinde J, van den Berg H A, Skowera A, Miles J J, et al., (2012) A single autoimmune T-cell receptor recognizes more than a million different peptides, J. Biol. Chem., 287: 1168-1177.
Yewdell J W (2010) Designing CD8⁺ T-cell vaccines: it's not rocket science (yet), Curr Opin Immunol, 22: 402-410.
Yewdell J W, Haeryfar S M (2005) Understanding presentation of viral antigens to CD8+ T-cells in vivo: the key to rational vaccine design, Annu. Rev. Immunol. 23: 651-682.
Zhao L, Lu W (2014) Mirror image proteins. Curr. Opin. Chem. Biol. 22: 56-61.
Martinez-Rodriguez S, Martinez-Gomez A I, Rodriguez-Vico F, Clemente-Jimenez J M, Las Heras-Vazquez F J (2010) Natural occurrence and industrial applications of D-amino acids: an overview. Chem. Biodivers. 7: 1531-1548.

1. A combinatorial peptide library (CPL) comprising a plurality of peptide sets, each set interrogating a different amino acid position (“index position”) of the peptide and comprising a plurality of separate peptide mixtures, wherein each mixture has a single defined D-amino acid (i.e., a, c, d, e, i, f, g, h, k, l, m, n, p, q, r, s, t, v, w, y, or modified forms thereof) at the index position, and with every other position being a random D-amino acid, wherein the number of sets is equal to the number of positions present in the peptides.

2. The CPL of claim 1, wherein the peptide mixture comprises an about equal representation of each amino acid at each position other than the index position.

3. The CPL of claim 1, wherein the D-amino acids in positions other than the index position exclude cysteine.

4. The CPL of claim 2, wherein each peptide in the mixture is present at approximately equimolar concentrations.

5. The CPL of claim 1, wherein the peptides are of a length known to be presented by MHC molecules.

6. The CPL of claim 5, wherein the MHC molecule is an MHC class I molecule.

7. The CPL of claim 1, wherein the peptides are synthesized by solid phase peptide synthesis (SPPS).

8. The CPL of claim 1, wherein each mixture is located in a separate well of a multi-well plate.

9. The CPL of claim 1, wherein the peptide mixtures are contacted with MHC molecules.

10. A method of identifying T-cell peptide agonists, the method comprising:
providing a combinatorial peptide library (CPL) to a MHC class I molecule known to interact with a T-cell receptor (TCR) of interest, the CPL comprising a plurality of peptide sets, each set interrogating a different amino acid position (“index position”) of the peptide and comprising a plurality of separate peptide mixtures, wherein each mixture has a single defined D-amino acid (i.e., a, c, d, e, f, g, h, k, l, m, n, p, q, r, s, t, v, w, y, or modified forms thereof) at the index position, and with every other position being a random D-amino acid, wherein the number of sets is equal to the number of positions present in the peptides;
contacting each MEW and peptide mixture with a T-cell receptor (TCR) known to bind the target antigen, to determine the D-amino acids recognized at each index position of the peptide;
generating an amino acid sequence by selecting recognized D-amino acids at each index position, to thereby identify T-cell agonists that elicit or enhance an immune response to the target antigen.

11. The method of claim 10, wherein the MEW molecule is present on the cell surface of an antigen presenting cell (APC).

12. The method of claim 10, wherein the TCR is presented on the surface of an effector cell.

13. The method of claim 12, wherein the effector cell is a T-cell that is known to recognize the target antigen.

14. The method of claim 10, wherein the peptide mixture is contacted with the TCR at a concentration of between about 1 μM and about 500 μM.

15. The method of claim 14, wherein the peptide mixture is contacted with the TCR at a concentration of about 100 μM.

16. The method of claim 10, wherein the determination of TCR recognition of each D-amino acid is selected based on effector cell function.

17. The method of claim 16, wherein the effector cell is a target antigen-specific cytotoxic T lymphocyte (CTL).

18. The method of claim 16, wherein the effector cell function is determined by a cytotoxicity assay, or the measurement of cytokine and/or chemokine release.

19. The method of claim 18, wherein the cytokine and/or chemokine release are measured using ELISA, ELISPOT, MHC-peptide tetramer staining, or flow cytometry.

20. The method of claim 18 or claim 19, wherein the cytokines and/or chemokines are selected from the group comprising: MIP-1α, MIP-1β, IFN-γ, IL-1, IL-2, IL-8, IL-12, IL-18, TFNβ, CD107a, and RANTES.

21. The method of claim 18, wherein the cytotoxicity assay is a chromium release assay.

22. The method of claim 10, wherein the screening further comprises the step of testing the identified D-amino acid peptide for immunogenicity.

23. An immunomodulating D-amino acid peptide that comprises, consists of, or consists essentially of an amino acid sequence represented by formula I:
X₁X₂pX₃X₄nnpp (I)
wherein:
X₁ is selected from g or r;
X₂ is selected from p or f;
X₃ is selected from p or q; and
X₄ is selected from w or g.

24. The immunomodulating D-amino acid peptide of claim 23, comprising, consisting, or consisting essentially of any one of the D-amino acid sequences set forth in SEQ ID NO: 3-10.

25. The immunomodulating D-amino acid peptide of claim 23, wherein the D-amino acid sequence comprises the sequence set forth in SEQ ID NO: 3.

26. A composition for eliciting or enhancing an immune response to a target antigen comprising, consisting, or consisting essentially of a D-amino acid peptide with an amino acid sequence represented by formula I:
X₁X₂pX₃X₄nnpp (I)
wherein:
X₁ is selected from g or r;
X₂ is selected from p or f;
X₃ is selected from p or q; and
X₄ is selected from w or g;
and wherein the target antigen is the influenza matrix M1 protein.

27. The composition of claim 26, comprising an amino acid sequence set forth in any one of SEQ ID NO: 3-10.

28. The composition of claim 26, wherein the D-amino acid peptide comprises the sequence set forth in SEQ ID NO: 3.

29. The composition of claim 26, wherein the composition is in particulate form.

30. A pharmaceutical composition comprising the D-amino acid peptide of claim 23, and a pharmaceutically acceptable carrier, diluent and/or excipient.

31. The pharmaceutical composition of claim 30, formulated as a vaccine.

32. The composition of claim 26, wherein the composition is formulated for oral administration.

33. A method for treating influenza, the method comprising administering a D-amino acid peptide antigen to a subject, wherein the D-amino acid peptide comprises, consists of, or consists essentially of an amino acid sequence represented by formula I:
X₁X₂pX₃X₄nnpp (I)
wherein:
X₁ is selected from g or r;
X₂ is selected from p or f;
X₃ is selected from p or q; and
X₄ is selected from w or g.

34. The method of claim 33, wherein the amino acid sequence comprises, consists, or consists essentially of a sequence set forth in any one of SEQ ID NO: 3-10.

35. The method of claim 33, wherein the amino acid sequence comprises, consists, or consists essentially of the sequence set forth in SEQ ID NO: 3.

36. A pharmaceutical composition comprising the composition of claim 26, and a pharmaceutically acceptable carrier, diluent and/or excipient.

37. The pharmaceutical composition of claim 36, formulated as a vaccine.
